(19) United States
(12) Patent Application Publication
(10) Pub. No.: US 2012/0070862 A1
(43) Pub. Date: Mar. 22, 2012

(54) METHODS FOR PRODUCING UNIQUELY DISTINCT NUCLEIC ACID TAGS

(75) Inventors: Nelson Alexander, Marana, AZ (US); Stacey Stanislaw, Tucson, AZ (US)

(73) Assignee: Ventana Medical Systems, Inc.

(21) Appl. No.: 13/289,702

(22) Filed: Nov. 4, 2011

Related U.S. Application Data
(63) Continuation-in-part of application No. 12/930,172, filed on Dec. 30, 2010.
(60) Provisional application No. 61/291,750, filed on Dec. 31, 2009.
(60) Provisional application No. 61/314,654, filed on Mar. 17, 2010.

(30) Foreign Application Priority Data
Dec. 30, 2010 (US) PCT/US2010/062485

(51) Int. Cl. C07H 21/00; C12P 19/34; C07H 1/00

(52) U.S. Cl. 435/91.52; 536/25.3

(57) ABSTRACT

Disclosed herein are uniquely distinct nucleic acid tags and methods for their use and production. The disclosed tags do not hybridize to a genome of interest and thus can be used as labels without generating background signal associated with unintended hybridization. In one example, tag sequences are derived from a genome divergent to the genome of interest. The divergent genome provides a vast library of potential tag sequences. These potential tag sequences can be screened using a bioinformatics-based approach against the genome of interest. These potentially distinct sequences can then be synthesized and tested empirically against the genome of interest to identify those sequences that are uniquely distinct. The tags can then be produced, for example by oligonucleotide synthesis techniques.

### CROSS REFERENCE TO RELATED APPLICATIONS

This is a continuation-in-part of U.S. patent application Ser. No. 12/930,172, filed Dec. 30, 2010, which in turn claims the benefit of U.S. Provisional Application No. 61/291,750, filed Dec. 31, 2009, and U.S. Provisional Application No. 61/314,654, filed Mar. 17, 2010, all of which are incorporated herein by reference in their entirety. This application is also related to International Application No. PCT/US2010/62485, filed Dec. 30, 2010, incorporated herein by reference.

### FIELD

This disclosure relates to the field of producing nucleic acid probes and tags. More specifically, this disclosure relates to methods for producing uniquely specific nucleic acid probes and uniquely distinct tags, and tags and probes generated by the disclosed methods. The uniquely specific nucleic acid sequences are in some examples represented only once in the haploid genome of an organism and the uniquely distinct tags are absent in the haploid genome of an organism of interest.

### BACKGROUND

Molecular cytogenetic techniques, such as fluorescence in situ hybridization (FISH), chromogenic in situ hybridization (CISH) and silver in situ hybridization (SISH), combine visual evaluation of chromosomes (karyotypic analysis) with molecular techniques. Molecular cytogenetics methods are based on hybridization of a nucleic acid probe to its complementary nucleic acid within a cell. A probe for a specific chromosomal region will recognize and hybridize to its complementary sequence on a metaphase chromosome or within an interphase nucleus (for example in a tissue sample). Probes have been developed for a variety of diagnostic and research purposes. For example, certain probes produce a chromosome banding pattern that mimics traditional cytogenetic staining procedures and permits identification of individual chromosomes for karyotypic analysis. Other probes are derived from a single chromosome and when labeled can be used as “chromosome paints” to identify specific chromosomes within a cell. Yet other probes identify particular chromosome structures, such as the centromeres or telomeres of chromosomes. Additional probes hybridize to single copy DNA sequences in a specific chromosomal region or gene. These are the probes used to identify the critical chromosomal region or gene associated with a syndrome or condition of interest. On metaphase chromosomes, such probes hybridize to each chromatid, usually giving two small, discrete signals per chromosome.

Hybridization of such chromosomal or gene-specific probes has made possible detection of chromosomal abnormalities associated with numerous diseases and syndromes, including constitutive genetic anomalies, such as microdeletion syndromes, chromosome translocations, gene amplification and aneuploidy syndromes, neoplastic diseases, as well as pathogen infections. Most commonly these techniques are applied to standard cytogenetic preparations on microscope slides. In addition, these procedures can be used on slides of formalin-fixed tissue, blood or bone marrow smears, and directly fixed cells or other nuclear isolates. Chromosomal or gene-specific probes can also be used in comparative genomic hybridization (CGH) to determine gene copy number in a genome.

The genome of many organisms contains repetitive nucleic acid sequences, which are series of nucleotides that are repeated multiple times, often in tandem arrays. The presence of such repetitive sequences in a probe results in increased background staining and requires the use of blocking DNA during hybridization. “Repeat-free” probes which lack such repetitive sequences are often generated (for example using a computer algorithm) to reduce this problem. However, even “repeat-free” probes require the use of substantial amounts of blocking DNA in order to reduce background staining to acceptable levels.

### SUMMARY

Disclosed herein are uniquely specific nucleic acid probes and methods for their use and production. The disclosed probes have reduced or eliminated background signal while reducing or eliminating the use of blocking DNA during hybridization. In some examples, probes are produced by a method that includes joining at least a first binding region and a second binding region in a pre-determined order and orientation, wherein the first binding region and second binding region are complementary to uniquely specific nucleic acid sequences, wherein the uniquely specific nucleic acid sequences are represented only once in a genome of an organism and wherein the first binding region and the second binding region include about 20% or less (for example 20%, 19%, 18%, 17%, 16%, 15%, 14%, 13%, 12%, 11%, 10%, 9%, 8%, 7%, 6%, 5%, 4%, 3%, 2%, 1%, or less) of a genomic target nucleic acid molecule. In some examples, the first binding region and the second binding region include about 10% or less of a genomic target nucleic acid molecule. In particular examples, the binding regions (“uniquely specific binding regions”) are complementary to non-contiguous portions of the genomic target nucleic acid. In some examples, the uniquely specific binding regions are at least about 20 base pairs (bp) in length (for example, about 35-500 bp, such as about 100 bp). In some examples, the genomic target nucleic acid is from a eukaryotic genome (such as a mammalian genome, for example a human genome).

In particular embodiments, the uniquely specific binding regions are generated by one or more of the following: separating the genomic target nucleic acid into a plurality of segments (for example, separating the genomic nucleic acid sequence into segments, such as in silico); comparing each segment with a genome including the genomic target nucleic acid (for example, using a computer algorithm, such as BLAT); selecting at least two segments which are uniquely specific to the genomic target nucleic acid (such as at least two segments that are each represented only once each in the genomic target nucleic acid molecule); removing repetitive DNA sequences from the genomic target nucleic acid (for example, using a computer algorithm, such as RepeatMasker); and selecting at least two segments having a GC nucleotide content between about 30% and 70%.

In other embodiments, the uniquely specific binding regions are generated by one or more of the following: separating the genomic target nucleic acid into a plurality of segments (for example, separating the genomic nucleic acid sequence into segments, such as in silico); synthesizing the plurality of nucleic acid segments; attaching the synthesized plurality of nucleic acid segments to an array; hybridizing the array with total genomic DNA and blocking DNA; selecting at least two segments which are uniquely specific to the genomic target nucleic acid (such as at least two segments that are each represented only once each in the genomic target nucleic acid molecule); removing repetitive DNA sequences from the genomic target nucleic acid (for example, using a computer algorithm, such as RepeatMasker); and selecting at least two segments having a GC nucleotide content between about 30% and 70%.

In some examples, the uniquely specific binding regions are generated by synthesizing a plurality of nucleic acid segments including the target genomic region, attaching the synthesized plurality of nucleic acid segments to an array, hybridizing the array with total genomic DNA and blocking DNA, and selecting at least two segments which are uniquely specific to the genomic target nucleic acid (such as at least two segments that are each represented only one each in the genomic target nucleic acid molecule).

In some examples, the pre-determined order and orientation is generated by the following: ordering the selected uniquely specific binding regions to produce a candidate nucleic acid probe (for example, ordering in the chromosomal order and orientation); separating the candidate nucleic acid probe into a plurality of segments (for example, separating the genomic nucleic acid sequence into segments, such as in silico); comparing each segment with a genome including the genomic target nucleic acid (for example, using a computer algorithm, such as BLAT); selecting at least one order and orientation of the selected segments that is uniquely specific to the genomic target nucleic acid (for example, does not include any sequence represented more than once in the genome of the organism); and joining the selected uniquely specific binding regions in the selected order and orientation. In other examples, the pre-determined order and orientation is generated by ordering the selected uniquely specific binding regions to produce a nucleic acid probe (for example in the chromosomal order and/or orientation) and joining the selected uniquely specific binding regions in the selected order and orientation.

Methods of using the disclosed probes include, for example, detecting (and in some examples quantifying) a genomic target nucleic acid sequence. For example, the method can include contacting the disclosed probes with a sample containing nucleic acid molecules under conditions sufficient to permit hybridization between the nucleic acid molecules in the sample and the plurality of nucleic acid molecules of the probe. Resulting hybridization is detected, wherein the presence of hybridization indicates the presence (and in some examples, the quantity) of the genomic target nucleic acid sequence.

Also disclosed are methods for producing nucleic acid tags, and tags produced using such methods. In some embodiments, the method includes selecting a prospect nucleic acid sequence from a first genomic sequence, the first genomic sequence corresponding to genomic DNA for a divergent organism. The prospect nucleic acid sequence is separated into a plurality of segment sequences, and the plurality of segment sequences compared to a second genomic sequence, the second genomic sequence corresponding to genomic DNA for an organism of interest. A plurality of segment sequences not homologous to any region of the second genomic sequence are selected from the plurality of segment sequences. A plurality of test oligonucleotides corresponding to the plurality of segment sequences not homologous to any region of the second genomic sequence are prepared, and the hybridization of the plurality of test oligonucleotides tested against the genomic DNA for the organism of interest. A plurality of tag sequences identified in the hybridization testing as being uniquely distinct from the genomic DNA for the organism of interest are selected, and nucleic acid tags prepared using one or more of the plurality of nucleic acid tag sequences identified in the hybridization testing as uniquely distinct from the genomic DNA for the organism of interest.

Kits including the probes, tags, and/or reagents for producing or using the probes and tags are also disclosed.

The foregoing and other features will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows an example of a portion of a Met proto-oncogene genomic nucleic acid sequence (SEQ ID NO: 1) that is enumerated and separated into 100 bp fragments. The repetitive sequence is replaced with “n”, followed by replacement of the number of “n”s by their numerical value. For example, there were 38 “n”s that were replaced by “*38*” in the line labeled “600.”

FIG. 2A shows BLAT results for a non-uniquely specific 100 bp segment of human chromosome 7.

FIG. 2B shows BLAT results for a uniquely specific 100 bp segment of human chromosome 7.

FIG. 3 is a digital image of a dot blot of selected segments 185 to 271 of an exemplary Met proto-oncogene (MET) probe in the form of 100 bp oligonucleotides immobilized on a membrane and hybridized with a human DNA probe. The three spots in the bottom right of the membrane correspond to human DNA controls (1 ng, 10 ng, and 100 ng).

FIG. 4A is a digital image of MDA-361 cells comparing ISH using a repeat-free MET probe made using prior methods (human placental blocking DNA was included during hybridization) to ISH using a uniquely specific MET probe of the present disclosure. No human blocking DNA was included during the uniquely specific probe hybridization; however salmon sperm DNA was included in the hybridization to counteract background binding of nucleic acids to non-nucleic acid reaction components, for example. Detection was via SISH colorimetric detection.

FIG. 4B is a digital image of MDA-361 cells comparing ISH using a repeat-free IGF1R probe made using prior methods (human placental blocking DNA was included during hybridization) to ISH using a uniquely specific IGF1R probe of the present disclosure. Human placental blocking DNA (minimal amounts compared to the repeat-free probe hybridization) and salmon sperm DNA were included during the uniquely specific probe hybridization. Detection was via SISH colorimetric detection.

FIG. 5A is a pair of digital images showing ISH performed with uniquely specific IGF1R probes to IGF1R target nucleic acids in a lung cancer tissue sample with (left) and without (right) human placental blocking DNA.

FIG. 5B is a pair of digital images showing ISH performed with uniquely specific TS probes to TS target nucleic acids in a lung cancer tissue sample with (left) and without (right) human placental blocking DNA.

FIG. 5C is a pair of digital images showing ISH performed with uniquely specific MET probes to Met proto-oncogene target nucleic acids in a lung cancer tissue sample with (left) and without (right) human placental blocking DNA.

FIG. 5D is a pair of digital images showing ISH performed with uniquely specific KRAS probes to KRAS target nucleic acids in a lung cancer tissue sample with (left) and without (right) human placental blocking DNA.

FIG. 6A is a plot of signal from hybridization of sequences targeting the CCND1 gene analyzed using a NimbleGen array. Pass/Fail criteria were established by including a series of positive and negative controls and using the data to establish thresholds for cutoffs.

FIG. 6B is a plot of signal from hybridization of sequences targeting the CDK4 gene analyzed using a NimbleGen array. Pass/Fail criteria were established by including a series of positive and negative controls and using the data to establish thresholds for cutoffs.

FIG. 6C is a plot of signal from hybridization of sequences targeting the Myb gene analyzed using a NimbleGen array. Pass/Fail criteria were established by including a series of positive and negative controls and using the data to establish thresholds for cutoffs.

FIG. 7A is a digital image showing ISH performed with a uniquely specific CCND1 probe in a lung cancer tissue sample without human placental blocking DNA.

FIG. 7B is a digital image showing ISH performed with uniquely specific CDK4 probe in a lung cancer tissue sample without human placental blocking DNA.

FIG. 7C is a digital image showing ISH performed with uniquely specific Myb probe in a lung cancer tissue sample without human placental blocking DNA.

FIG. 8 is a digital image showing ISH performed with a uniquely specific EGFR probe in a lung cancer tissue sample without human placental blocking DNA and detected with tyramide signal amplification.

### SEQUENCE LISTING

Any nucleic acid and amino acid sequences listed herein or in the accompanying sequence listing are shown using standard letter abbreviations for nucleotide bases, and three letter code for amino acids, as defined in 37 C.F.R. §1.822. In at least some cases, only one strand of each nucleic acid sequence is shown, but the complementary strand is understood as included by any reference to the displayed strand.

The Sequence Listing is submitted as an ASCII text file in the form of the file named Sequence_Listing.txt, which was created on Nov. 2, 2011, and is 2,058 bptes, which is incorporated by reference herein.

SEQ ID NO: 1 is an exemplary enumerated and separated Met proto-oncogene genomic sequence wherein repetitive sequences are replaced with “n.”

### DETAILED DESCRIPTION

### I. Introduction

Production of probes corresponding to selected target nucleic acid sequences (e.g., genomic target nucleic acid sequences) for molecular analysis can be complicated by the presence of undesired sequences in the probe that can potentially increase the amount of background signal. Examples of undesired sequences include, but are not limited to, interspersed repetitive nucleic acid elements present throughout eukaryotic (e.g., human) genomes and nucleic acid sequences that are present more than once in a genome (e.g. a “non-unique” sequence).

Historically, the selection of probes typically attempts to balance the strength of a target specific signal against the level of non-specific background. For example, in previous methods, when selecting a probe corresponding to a target, signal is generally maximized by increasing the sequence content of the probe. However, as the sequence content of a probe (e.g., for genomic target nucleic acid sequences) increases, so does the amount of undesired (e.g., repetitive and/or non-unique) nucleic acid sequence included in the probe. Attempts to increase the specificity of probes by decreasing the sequence content of the probe does not eliminate the inclusion of DNA sequences that maintain non-unique nucleic acid sequences that exist multiple times in the genome of interest (for example, the human genome). Such probes can contain sequences that are present numerous times (for example, up to 150-200 times) in the genome.

When the probe is labeled (either directly with a detectable moiety, such as a fluorophore, or indirectly with a moiety such as a hapten, which can be indirectly detected based on binding and detection of additional components), the undesired (e.g., repetitive and/or non-unique) nucleic acid sequence elements are labeled along with the target-specific elements within the target sequence. During hybridization, binding of the labeled undesired (e.g., repetitive and/or non-unique) nucleic acid sequences results in a dispersed background signal, which can confound interpretation, for example when numerical or quantitative data (such as copy number of a sequence or copy number difference between genomes) is desired. Reduction of background due to hybridization of labeled repetitive or other undesired nucleic acid sequences in the probe has typically been accomplished by adding blocking DNA (e.g., unlabeled repetitive DNA, such as Cot-1™ DNA or total genomic DNA) to the hybridization reaction.

The present disclosure provides an approach to reducing or eliminating background signal due to the presence of repetitive or other undesired (e.g. non-unique) nucleic acid sequences in a probe. In particular, the present disclosure provides probes and methods of producing probes that have reduced or eliminated background signal while reducing or eliminating the use of blocking DNA (such as human blocking DNA, for example, human placental DNA) and methods for producing such probes. Some exemplary probes disclosed herein are substantially or entirely free of repetitive or other non-unique nucleic acid sequences, such as probes that include substantially only uniquely specific nucleic acid sequences (for example, sequences that are represented in a genome only once).

Also provided are uniquely distinct nucleic acid tags and methods for their use and production. Such tags do not hybridize to a genome of interest (such as a human genome) and thus can be used as labels without generating background signal associated with unintended hybridization.

### II. Abbreviations

aCGH: array comparative genomic hybridization
BLAT: BLAST-like alignment tool
bp: base pair(s)
CCND1: cyclin D1
CDK4: cyclin-dependent kinase 4
CGH: comparative genomic hybridization
CISH: chromogenic in situ hybridization
EGFR: epidermal growth factor receptor
FISH: fluorescent in situ hybridization
IGF1R: insulin-like growth factor 1 receptor
ISH: in situ hybridization
MET: Met proto-oncogene (also known as hepatocyte growth factor receptor)
SISH: silver in situ hybridization

### III. Terms

Unless otherwise noted, technical terms are used according to conventional usage. Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); and George P. Redei, Encyclopedic Dictionary of Genetics, Genomics, and Proteomics, 2nd Edition, 2003 (ISBN: 0-471-26821-6).

The following explanations of terms and methods are provided to better describe the present disclosure and to guide those of ordinary skill in the art to practice the present disclosure. The singular forms “a,” “an,” and “the” refer to one or more than one, unless the context clearly dictates otherwise. For example, the term “comprising a cell” includes single or plural cells and is considered equivalent to the phrase “comprising at least one cell.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

All publications, patent applications, patents, and other references mentioned herein are incorporated by reference in their entirety for all purposes. All sequences associated with the GenBank Accession Nos. mentioned herein are incorporated by reference in their entirety as were present on Dec. 31, 2009, to the extent permissible by applicable rules and/or law. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting.

To facilitate review of the various embodiments of this disclosure, the following explanations of specific terms are provided:

Array: An arrangement of molecules, such as biological macromolecules (such as peptides or nucleic acid molecules) or biological samples (such as tissue sections), in addressable locations on or in a substrate. A “microarray” is an array that is miniaturized so as to require or be aided by microscopic examination for evaluation or analysis. Arrays are sometimes called chips or biochips.

The array of molecules (“features”) makes it possible to carry out a very large number of analyses on a sample at one time. In certain example arrays, one or more molecules (such as a nucleic acid molecule) will occur on the array a plurality of times (such as twice), for instance to provide internal controls. The number of addressable locations on the array can vary, for example from at least one, to at least 2, to at least 5, to at least 10, at least 20, at least 30, at least 50, at least 75, at least 100, at least 150, at least 200, at least 300, at least 500, least 550, at least 600, at least 800, at least 1000, at least 10,000, or more. In particular examples, an array includes nucleic acid molecules, such as nucleic acid molecules that are at least 20 nucleotides in length, such as about 20-500 nucleotides in length. In particular examples, an array includes nucleic acid molecules generated by separating a genomic target nucleic acid into a plurality of segments, for example using the methods provided herein.

Within an array, each arrayed sample is addressable, in that its location can be reliably and consistently determined within at least two dimensions of the array. The feature application location on an array can assume different shapes. For example, the array can be regular (such as arranged in uniform rows and columns) or irregular. Thus, in ordered arrays the location of each sample is assigned to the sample at the time when it is applied to the array, and a key may be provided in order to correlate each location with the appropriate target or feature position. Often, ordered arrays are arranged in a symmetrical grid pattern, but samples could be arranged in other patterns (such as in radially distributed lines, spiral lines, or ordered clusters). Addressable arrays usually are computer readable, in that a computer can be programmed to correlate a particular address on the array with information about the sample at that position (such as hybridization or binding data, including for instance signal intensity). In some examples of computer readable formats, the individual features in the array are arranged regularly, for instance in a Cartesian grid pattern, which can be correlated to address information by a computer.

In some examples, the array includes positive controls, negative controls, or both, for example nucleic acid molecules specific for known repetitive elements or nucleic acid molecules specific for an unrelated genome or organism. In one example, the array includes 1 to 100 controls, such as 1 to 60 or 1 to 20 controls.

Binding or stable binding: The association between two substances or molecules, such as the hybridization of one nucleic acid molecule (e.g., a binding region) to another (or itself) (e.g., a target nucleic acid molecule). A nucleic acid molecule (such as a binding region) binds or stably binds to a target nucleic acid molecule if a sufficient amount of the nucleic acid molecule forms base pairs or is hybridized to its target nucleic acid molecule to permit detection of that binding.

Binding can be detected by any procedure known to one skilled in the art, such as by physical or functional properties of the target:binding region complex. Physical methods of detecting the binding of complementary strands of nucleic acid molecules include, but are not limited to, such methods as DNase I or chemical footprinting, gel shift and affinity cleavage assays, Northern blotting, dot blotting and light absorption detection procedures. In another example, the method involves detecting a signal, such as a detectable label, present on one or both nucleic acid molecules (e.g., a label associated with the binding region).

Binding region: A segment or portion of a target nucleic acid molecule (for example, at least 20 bp, such as about 20-500 bp, or about 100 bp) that is uniquely specific to the target molecule. The nucleic acid sequence of a binding region and its corresponding target nucleic acid molecule have sufficient nucleic acid sequence complementarity such that when the two are incubated under appropriate hybridization conditions, the two molecules will hybridize to form a detectable complex. A target nucleic acid molecule can contain multiple different binding regions, such as at least 10, at least 50, at least 100, at least 1000, at least 1500 or more unique binding regions. In particular examples, a binding region is approximately 20 to 500 bp in length. When obtaining binding regions from a target nucleic acid sequence, the target sequence can be obtained in its native form in a cell, such as a mammalian cell, or in a cloned form (e.g., in a vector).

Complementary: A nucleic acid molecule is said to be complementary with another nucleic acid molecule if the two molecules share a sufficient number of complementary nucleotides to form a stable duplex or triplex when the strands bind (hybridize) to each other, for example by forming Watson-Crick, Hoogsteen, or reverse Hoogsteen base pairs. Stable binding occurs when a nucleic acid molecule (e.g., a uniquely specific nucleic acid molecule) remains detectably bound to a target nucleic acid (e.g., genomic target nucleic acid) under the required conditions.

Complementarity is the degree to which bases in one nucleic acid molecule (e.g., a probe nucleic acid molecule) base pair with the bases in a second nucleic acid molecule (e.g., genomic target nucleic acid molecule). Complementarity is conveniently described by percentage, that is, the proportion of nucleotides that form base pairs between two molecules or within a specific region or domain of two molecules. For example, if 10 nucleotides of a 15 contiguous nucleotide region of a probe nucleic acid molecule form base pairs with a target nucleic acid molecule, that region of the probe nucleic acid molecule is said to have 66.67% complementarity to the target nucleic acid molecule.

In the present disclosure, “sufficient complementarity” means that a sufficient number of base pairs exist between one nucleic acid molecule or region thereof (such as a uniquely specific binding region) and a target nucleic acid sequence (e.g., genomic target nucleic acid sequence) to achieve detectable binding. A thorough treatment of the qualitative and quantitative considerations involved in establishing binding conditions is provided by Beltz et al. Methods Enzymol. 100:266-285, 1983, and by Sambrook et al. (ed.), Molecular Cloning: A Laboratory Manual, 2nd ed., vol. 1-3, Cold Spring Harbor Laboratory Press, Cold Spring Harbor, N.Y., 1989.

Computer implemented algorithm: An algorithm or program (set of executable code in a computer readable medium) that is performed or executed by a computing device at the command of a user. In the context of the present disclosure, computer implemented algorithms can be used to facilitate (e.g., automate) selection of polynucleotide sequences with particular characteristics, such as identification of uniquely specific nucleic acid sequences of a target nucleic acid sequence. Typically, a user initiates execution of the algorithm by inputting a command, and setting one or more selection criteria, into a computer, which is capable of accessing a sequence database. The sequence database can be encompassed within the storage medium of the computer or can be stored remotely and accessed via a connection between the computer and a storage medium at a nearby or remote location via an intranet or the internet. Following initiation of the algorithm, the algorithm or program is executed by the computer, e.g., to compare one or more segments of a target nucleic acid with the genome comprising the target nucleic acid molecule. Most commonly, the results of the comparison are then displayed (e.g., on a screen) or outputted (e.g., in printed format or onto a computer readable medium).

Detectable label: A compound or composition that is conjugated directly or indirectly to another molecule (such as a uniquely specific nucleic acid molecule) to facilitate detection of that molecule. Specific, non-limiting examples of labels include fluorescent and fluorogenic moieties, chromogenic moieties, haptens, affinity tags, and radioactive isotopes. The label can be directly detectable (e.g., optically detectable) or indirectly detectable (for example, via interaction with one or more additional molecules that are in turn detectable). Exemplary labels in the context of the probes disclosed herein are described below. Methods for labeling nucleic acids, and guidance in the choice of labels useful for various purposes, are discussed, e.g., in Sambrook and Russell, in Molecular Cloning: A Laboratory Manual, 3^{rd} Ed., Cold Spring Harbor Laboratory Press (2001) and Ausubel et al., in Current Protocols in Molecular Biology, Greene Publishing Associates and Wiley-Intersciences (1987, and including updates).

DNA blocking reagent: A preparation of genomic DNA (such as human genomic DNA, for example human placental DNA) that is included in a hybridization reaction to decrease binding (for example, hybridization) of a nucleic acid probe to non-target nucleic acids (e.g., repetitive nucleic acid sequences) in a sample. In some examples, a blocking reagent is unlabeled repetitive DNA, for example, Cot-1™ Blocking DNA is distinguished from carrier DNA (such as salmon sperm DNA or herring sperm DNA), which is included in a hybridization reaction to reduce non-specific binding of a probe to non-nucleic acid components (for example, a tube, slide, membrane, protein, or other non-nucleic acid component that a probe contacts during experimental handling).

Genome: The total genetic constituents of an organism. In the case of eukaryotic organisms, the genome is contained in a haploid set of chromosomes of a cell. The genome of an organism may also include non-chromosomal DNA, such as mitochondrial DNA or chloroplast DNA. In particular examples, a genome is a mammalian genome (for example, a human genome).

Hybridization: To form base pairs between complementary regions of two strands of DNA, RNA, or between DNA and RNA, thereby forming a duplex molecule. Hybridization conditions resulting in particular degrees of stringency will vary depending upon the nature of the hybridization method and the composition and length of the hybridizing nucleic acid sequences. Generally, the temperature of hybridization and the ionic strength (such as the Na⁺ concentration) of the hybridization buffer will determine the stringency of hybridization. The presence of a chemical which decreases hybridization (such as formamide) in the hybridization buffer will also determine the stringency (Sadhu et al., J. Biosci. 6:817-821, 1984). Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11). Hybridization conditions for ISH are also discussed in Landegent et al., Hum. Genet. 77:366-370, 1987; Lichter et al., Hum. Genet. 80:224-234, 1988; and Pinkel et al., Proc. Natl. Acad. Sci. USA 85:9138-9142, 1988.

Isolated: An “isolated” biological component (such as a nucleic acid molecule, protein, or cell) has been substantially separated or purified away from other biological components in the cell of the organism, or the organism itself, in which the component naturally occurs, such as other chromosomal and extra-chromosomal DNA and RNA, proteins and cells. Nucleic acid molecules and proteins that have been “isolated” include nucleic acid molecules and proteins purified by standard purification methods. The term also embraces nucleic acid molecules and proteins prepared by recombinant expression in a host cell as well as chemically synthesized nucleic acid molecules and proteins.

Joined or joining: Physically connected or linked. In particular examples, the binding regions (such as uniquely specific binding regions) described herein are joined or linked together to produce a uniquely specific probe. Typically the binding regions are joined enzymatically by a ligase in a ligation reaction.

However, binding regions can also be joined chemically, for example, by incorporating appropriate modified nucleotides (as described in Dolinnaya et al., Nucleic Acids Res. 16:3721-38, 1988; Mattes and Seitz, Chem. Commun. 2050-2051, 2001; Mattes and Seitz, Agnew. Chem. Int. 40:3178-81, 2001; Ficht et al., J. Am. Chem. Soc. 126:9970-81, 2004) or by chemical synthesis of the polynucleotide including the binding regions. Alternatively, two binding regions can be joined in an amplification reaction, or using a recombinase.

Nucleic acid: A deoxyribonucleotide or ribonucleotide polymer in either single or double stranded form, and unless otherwise limited, encompassing analogs of natural nucleotides that hybridize to nucleic acids in a manner similar to naturally occurring nucleotides. The term “nucleotide” includes, but is not limited to, a monomer that includes a base (such as a pyrimidine, purine or synthetic analogs thereof) linked to a sugar (such as ribose, deoxyribose or synthetic analogs thereof), or a base linked to an amino acid, as in a peptide nucleic acid (PNA). A nucleotide is one monomer in a polynucleotide. A nucleotide sequence refers to the sequence of bases in a polynucleotide.

A nucleic acid “segment” is a subportion or subsequence of a target nucleic acid molecule. A nucleic acid segment can be derived hypothetically or actually from a target nucleic acid molecule in a variety of ways. For example, a segment of a target nucleic acid molecule (such as a genomic target nucleic acid molecule) can be obtained by digestion with one or more restriction enzymes to produce a nucleic acid segment that is a restriction fragment. Nucleic acid segments can also be produced from a target nucleic acid molecule by amplification, by hybridization (for example, subtractive hybridization), by artificial synthesis, or by any other procedure that produces one or more nucleic acids that correspond in sequence to a target nucleic acid molecule. Nucleic acid segments may also be produced in silico, for example using a computer-implemented algorithm. A particular example of a nucleic acid segment is a binding region.

Probe: A nucleic acid molecule that is capable of hybridizing with a target nucleic acid molecule (e.g., genomic target nucleic acid molecule) and, when hybridized to the target, is capable of being detected either directly or indirectly. Thus probes permit the detection, and in some examples quantification, of a target nucleic acid molecule. In particular examples, a probe includes at least two binding regions, such as two or more binding regions complementary to uniquely specific nucleic acid sequences of a target nucleic acid molecule and are thus capable of specifically hybridizing to at least a portion of the target nucleic acid molecule. Generally, once at least one binding region or portion of a binding region has (and remains) hybridized to the target nucleic acid molecule other portions of the probe may (but need not) be physically constrained from hybridizing to those other portions' cognate binding sites in the target (e.g., such other portions are too far distant from their cognate binding sites); however, other nucleic acid molecules present in the probe can bind to one another, thus amplifying signal from the probe. A probe can be referred to as a “labeled nucleic acid probe,” indicating that the probe is coupled directly or indirectly to a detectable moiety or “label,” which renders the probe detectable.

Repeat-free sequence: A nucleic acid that does not include an appreciable amount of repetitive nucleic acid (e.g., DNA) sequences or “repeats.” However, in some examples, “repeat-free” sequences may still include one or more nucleic acid segments including repetitive nucleic acid sequences or having homology or sequence identity to multiple portions of the genome. Repetitive nucleic acid sequences are nucleic acid sequences within a nucleic acid (such as a genome, for example a mammalian genome) which encompass a series of nucleotides which are repeated many times, often in tandem arrays. The repetitive nucleic acid sequences can occur in a nucleic acid sequence (e.g., a mammalian genome) in multiple copies ranging from two to hundreds of thousands of copies, and can be clustered or interspersed on one or more chromosomes throughout a genome. In some examples, the presence of significant repetitive nucleic acid sequences in a probe can increase background signal. Repetitive nucleic acid sequences include, but are not limited to for example in humans, telomere repeats, subtelomeric repeats, microsatellite repeats, minisatellite repeats, Alu repeats, L1 repeats, Alpha satellite DNA, and satellite 1, H, and III repeats.

Sample: A biological specimen containing DNA (for example, genomic DNA), RNA (including mRNA), protein, or combinations thereof, obtained from a subject. Examples include, but are not limited to, chromosomal preparations, peripheral blood, urine, saliva, tissue biopsy, surgical specimen, bone marrow, amniocentesis samples, and autopsy material. In one example, a sample includes genomic DNA. In some examples, the sample is a cytogenetic preparation, for example which can be placed on microscope slides. In particular examples, samples are used directly, or can be manipulated prior to use, for example, by fixing (e.g., using formalin).

Sequence identity: The identity (or similarity) between two or more nucleic acid sequences is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biotechnology (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site.

BLASTN may be used to compare nucleic acid sequences, while BLASTP may be used to compare amino acid sequences. If the two compared sequences share homology, then the designated output file will present those regions of homology as aligned sequences. If the two compared sequences do not share homology, then the designated output file will not present aligned sequences.

The BLAST-like alignment tool (BLAT) may also be used to compare nucleic acid sequences (Kent, Genome Res. 12:656-664, 2002). BLAT is available from several sources, including Kent Informatics (Santa Cruz, Calif.) and on the Internet (genome.ucsc.edu).

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 15 consecutive nucleotides from an identified sequence as follows contains a region that shares 75 percent sequence identity to that identified sequence (that is, 15-20*100=75).

Subject: Any multi-cellular vertebrate organism, such as human and non-human mammals (e.g., veterinary subjects).

Target genome: A genome (such as a haploid or diploid genome) from an organism of interest. In some examples, the target genome is a genome including a target genomic nucleic acid molecule. In other examples, a target genome is a genome in which detection of a nucleic acid molecule is desired (for example by a hybridization assay). In one example, a target genome is a human genome.

Target nucleic acid sequence or molecule: A defined region or particular portion of a nucleic acid molecule, for example a portion of a genome (such as a gene or a region of mammalian genomic DNA containing a gene of interest). In an example where the target nucleic acid sequence is a target genomic sequence (such as a haploid or diploid genome), such a target can be defined by its position on a chromosome (e.g., in a normal cell), for example, according to cytogenetic nomenclature by reference to a particular location on a chromosome; by reference to its location on a genetic map; by reference to a hypothetical or assembled contig; by its specific sequence or function; by its gene or protein name; or by any other means that uniquely identifies it from among other genetic sequences of a genome. In some examples, the target nucleic acid sequence is mammalian genomic sequence (for example human genomic sequence).

In some examples, alterations of a target nucleic acid sequence (e.g., genomic nucleic acid sequence) are “associated with” a disease or condition. That is, detection of the target nucleic acid sequence can be used to infer the status of a sample with respect to the disease or condition. For example, the target nucleic acid sequence can exist in two (or more) distinguishable forms, such that a first form correlates with absence of a disease or condition and a second (or different) form correlates with the presence of the disease or condition. The two different forms can be qualitatively distinguishable, such as by polynucleotide polymorphisms, and/or the two different forms can be quantitatively distinguishable, such as by the number of copies of the target nucleic acid sequence that are present in a cell.

Uniquely distinct sequence: A nucleic acid sequence that is not present in a genome of an organism (such as a genome of interest), such as a sequence that is at least 12, at least 15, at least 20, at least 50, at least 100, or at least 500 nucleotides in length and is not present in a genome (such as a genome of interest). In a particular example, a uniquely distinct nucleic acid sequence is a nucleic acid sequence selected from a divergent organism's genome that has no significant identity to any nucleic acid sequences present in the genome of interest. In some examples, uniquely distinct nucleic acid sequences can be identified using a computer-implemented algorithm, for example, BLAT. In other examples, uniquely distinct nucleic acid sequences can be identified empirically, for example, using hybridization, or lack thereof, to nucleic acid sequences on an array.

Uniquely specific sequence: A nucleic acid sequence of any length that is present only one time in a genome of an organism. In a particular example, a uniquely specific nucleic acid sequence is a nucleic acid sequence from a target nucleic acid that has 100% sequence identity with the target nucleic acid and has no significant identity to any other nucleic acid sequences present in the specific genome that includes the target nucleic acid. In some examples, uniquely specific nucleic acid sequences can be identified using a computer-implemented algorithm, for example, BLAT. In other examples, uniquely specific nucleic acid sequences can be identified empirically, for example, using hybridization to nucleic acid sequences on an array.

Vector: Any nucleic acid that acts as a carrier for other (“foreign”) nucleic acid sequences that are not native to the vector. When introduced into an appropriate host cell a vector may replicate itself (and, thereby, the foreign nucleic acid sequence) or express at least a portion of the foreign nucleic acid sequence. In one context, a vector is a linear or circular nucleic acid into which a nucleic acid sequence of interest is introduced (for example, cloned) for the purpose of replication (e.g., production) and/or manipulation using standard recombinant nucleic acid techniques (e.g., restriction digestion). A vector can include nucleic acid sequences that permit it to replicate in a host cell, such as an origin of replication. A vector can also include one or more selectable marker genes and other genetic elements known in the art. Common vectors include, for example, plasmids, cosmids, phage, phagemids, artificial chromosomes (e.g., BAC, PAC, HAC, YAC) and hybrids that incorporate features of more than one of these types of vectors. Typically, a vector includes one or more unique restriction sites (and in some cases a multi-cloning site) to facilitate insertion of a target nucleic acid sequence.

In one example discussed herein, two or more binding regions complementary to uniquely specific nucleic acid sequences are introduced and replicated in a vector, such as a plasmid or an artificial chromosome (e.g., yeast artificial chromosome, P1 based artificial chromosome, bacterial artificial chromosome (BAC)).

### IV. Methods for Producing Uniquely Specific Probes or Tags

Methods of producing nucleic acid probes including binding regions that are complementary to uniquely specific nucleic acid sequences of a target nucleic acid molecule are disclosed herein. In particular examples, the methods include joining at least a first binding region and a second binding region in a pre-determined order and orientation, wherein the binding regions are complementary to uniquely specific nucleic acid sequences (for example, sequences that are represented only once in a genome of an organism) and the binding regions include about 20% or less of a genomic target nucleic acid molecule.

In one example, at least two uniquely specific binding regions (such as at least 5, 10, 50, 100, 200, 300, 400, 500, 600, 700, 800, 900, 1000, 1200, 1500, 1800, 2000, 2500, 3000, or more binding regions) are included in a nucleic acid probe. In particular examples, about 200 to 3000 (such as about 300 to 600, about 350 to 550, about 500 to 600, or about 500 to 3000, about 500 to 2000, or about 2000 to 3000) uniquely specific binding regions are included in a nucleic acid probe.

In some examples the methods disclosed herein provide for generation of a nucleic acid probe that includes at least two binding regions complementary to uniquely specific nucleic acid sequences. Much of the genome of an organism (for example, a eukaryotic organism, such as a mammal, e.g., a human) consists of non-uniquely specific nucleic acid sequence (for example, repetitive sequence or sequences represented more than once in the genome). For example, the proportion of mammalian genome that consists of repetitive sequence is estimated to be approximately 40-50% (e.g., Lander et al., Nature 409:860-921, 2001). Thus, the portion of a genomic target nucleic acid molecule that is uniquely specific will be only a fraction of the target nucleic acid molecule. There are also regional differences within genomes, for example the human genome. For example, regional differences comprise differences between centromeric DNA, telomeric DNA, etc. In some examples, the binding regions selected for the probe are non-contiguous and/or are distributed throughout the genomic target nucleic acid molecule. In particular examples, the binding regions complementary to uniquely specific nucleic acid sequence represent less than about 20% (such as less than about 20%, 19%, 18%, 17%, 16%, 15%, 14%, 13%, 12%, 11%, 10%, 9%, 8%, 7%, 6%, 5%, 4%, 3%, 2%, 1%, or even less) of the genomic target nucleic acid molecule. For example, the binding regions complementary to uniquely specific nucleic acid sequence may represent about 1-20% (such as about 15-20%, about 10-15%, about 2-8%, about 3-6%, or about 2-3%) of the genomic target nucleic acid molecule.

Also provided are methods for producing nucleic acid tags, such as a method that includes selecting a prospect nucleic acid sequence from a first genomic sequence corresponding to genomic DNA for a divergent organism; separating the prospect nucleic acid sequence into a plurality of segment sequences; comparing the plurality of segment sequences to a second genomic sequence corresponding to genomic DNA for an organism of interest; selecting a plurality of segment sequences not homologous to any region of the second genomic sequence from the plurality of segment sequences; preparing a plurality of test oligonucleotides corresponding to the plurality of segment sequences not homologous to any region of the second genomic sequence; testing hybridization of the plurality of test oligonucleotides against the genomic DNA for the organism of interest; selecting a plurality of tag sequences identified in the hybridization testing as uniquely distinct from the genomic DNA for the organism of interest; and preparing the nucleic acid tags using one or more of the plurality of nucleic acid tag sequences identified in the hybridization testing as uniquely distinct from the genomic DNA for the organism of interest.

A. Identifying Uniquely Specific Sequences

In some examples the disclosed methods include identifying two or more nucleic acid segments that are uniquely specific to a target nucleic acid. A uniquely specific nucleic acid sequence is a nucleic acid sequence of at least 20 bp (such as at least 20 bp, 30 bp, 40 bp, 50 bp, 60 bp, 70 bp, 80 bp, 90 bp, 100 bp, or more) that is present only one time in the genome of the organism in which the target nucleic acid is present or from which the target nucleic acid is derived. For example, a uniquely specific nucleic acid sequence can be a nucleic acid sequence from a region of the target nucleic acid that has 100% sequence identity with that region of the target nucleic acid and has no significant identity to any other nucleic acid sequence in the genome which includes the target nucleic acid molecule.

In particular examples, a genomic target nucleic acid molecule or other nucleic acid molecule of interest is selected (such as one or more of those discussed in Section V, below). The nucleic acid sequence of the genomic target nucleic acid or other nucleic acid molecule is obtained, for example, by in silico methods (such as from a database) or by direct sequencing. In some examples, the genomic target nucleic acid or other nucleic acid molecule (for example, a eukaryotic gene target) includes at least about 10,000 bp, such as at least about 20,000, 30,000, 40,000, 50,000, 100,000, 250,000, 500,000, 600,000, 700,000, 800,000, 900,000, 1,000,000, 1,500,000, 2,000,000, 3,000,000, 4,000,000 bp, or more (such as an entire chromosome or even an entire genome).

Following selection of a genomic target nucleic acid or other nucleic acid sequence, repetitive sequences are optionally detected and removed from the sequence. In some examples, most or substantially all repetitive nucleic acid sequences (for example, substantially all known repeat sequences for the particular genome) are identified and removed from the sequence. For example, repetitive sequences (such as telomere repeats, subtelomeric repeats, microsatellite repeats, minisatellite repeats, Alu repeats, L1 repeats, Alpha satellite DNA, and satellite 1, H, and III repeats) can be identified using a computer implemented algorithm. Such algorithms are known in the art and include software applications such as RepeatMasker (available on the World Wide Web at repeatmasker.org) and CENSOR (Kohany et al., BMC Bioinformatics 7:474, 2006; available on the World Wide Web at girinst.org/censor/index.php). In a particular example, RepeatMasker is used to identify repetitive sequences. Once repetitive sequences are identified, they are removed from the genomic target nucleic acid sequence or other nucleic acid molecule, or “masked” (for example, the repetitive sequence may be replaced with a non-nucleotide character, such as “N” or with a number indicating the number of consecutive base pairs that are masked). Some computer algorithms for identifying repetitive nucleic acid sequences also “mask” the repetitive sequences (for example, RepeatMasker and CENSOR). This generates a substantially repeat-free genomic target nucleic acid sequence.

To facilitate the automation of sequence selection, in one example, the selected genomic target nucleic acid sequence or other nucleic acid molecule (such as a substantially repeat-free genomic target nucleic acid sequence or other nucleic acid molecule) is enumerated (numbered) and separated in silico into segments, such as segments of about 20-500 bp (for example, about 50-250 bp, about 75-250 bp, about 100-200 bp, about 250-500 bp, or about 35-50 bp). In a particular example, the segments are each about 100 bp. The genomic target nucleic acid sequence or other nucleic acid molecule may be enumerated and separated in non-overlapping, consecutive segments or into overlapping, consecutive segments (for example, overlapping by at least one base pair, such as 1, 2, 3, 4, 5, 10, 15, 20, 50, or more bp). In one example, the genomic target nucleic acid sequence or other nucleic acid molecule is separated into consecutive non-overlapping 100 base pair segments (for example, bases 1-100, 101-200, 201-300 of the genomic target nucleic acid sequence or other nucleic acid molecule, and so on). In another example, the genomic target nucleic acid sequence or other nucleic acid molecule is separated into consecutive 100 base pair segments that overlap by at least one base pair (such as overlap of 99, 98, 97, 96, 95, 90, 85, 80 base pairs, and so on), for example, bases 1-100, 2-101, 3-102, 4-103 and so on; or bases 1-100, 5-105, 10-110, and so on; or bases 1-100, 10-110, 20-120 of the genomic target nucleic acid sequence or other nucleic acid molecule, and so on. In a particular example, the genomic target nucleic acid sequence or other nucleic acid molecule is separated into consecutive 100 base pair segments that overlap by at least ten base pairs, such as bases 1-100, 10-110, 20-120, 30-130 of the genomic target nucleic acid sequence or other nucleic acid molecule, and so on.

One of skill in the art can select the amount of sequence overlap used in the disclosed methods, for example, based on the size of the target sequence or other sequence of interest or the amount of non-repetitive and/or unique sequence present in the sequence. In some examples, if the target sequence or other sequence of interest is relatively small or includes a high number of repetitive sequences, it may be desirable to utilize a larger overlap (for example, 100 bp segments that overlap by at least 99, 98, 97, 96, 95, 94, 93, 92, 91, or 90 base pairs). In other examples, if the target sequence or other sequence of interest is relatively large or contains a low number of repetitive sequences, a smaller overlap (for example, 100 bp segments that overlap by 10, 9, 8, 7, 6, 5, 4, 3, 2, or 1 base pairs) or no overlap may be utilized. In some examples, if a selected number of uniquely specific sequences from a genomic target region is not obtained with a particular overlap, the overlap amount is increased until the desired number of uniquely specific sequences from the genomic target region is obtained.

In other examples, the enumeration and separation of sequences are carried out using a computer implemented algorithm (for example, a macro-embedded word processing file). In one example, the MATLAB® programming language (version 7.9.0.529 (R2009b); The MathWorks, Inc., Natick, Mass.) is used to develop an algorithm to identify multiple 100 bp segments that are tiled (overlap) by at least one base pair (such as at least 1, 2, 3, 4, 5, 10, 15, 20, 50, or more base pairs). In another example, the enumeration and separation of sequences is carried out using a sliding window reading frame where every possible sequence of a selected length (such as 20-500 bp) is analyzed for any given target nucleic acid sequence.

In some examples, the nucleic acid segments are about 100 bp. For example, segments of about 20-500 bp can be used for the disclosed methods. Commonly used methods for probe labeling (such as nick translation) result in labeled fragments of approximately 100-500 bp. Thus, having uniquely specific segments of greater than about 500 bp may not improve probe signal strength. In addition, because the labeled probe fragments are generally longer than the uniquely specific nucleic acid sequences, each labeled fragment may contain multiple non-contiguous portions of the target nucleic acid sequence. This allows the probe fragments to form scaffolds, thereby increasing the signal strength of the probe. Having uniquely specific segments of about 20-500 bp also allows the probe to be spread out over the larger target nucleic acid sequence. In some examples, the selected uniquely specific segments are separated by at least about 100 bp to about 70,000 bp (such as at least about 200-50,000 bp, about 500-25,000 bp, about 1000-10,000 bp, or about 500-5000 bp) in the genomic target nucleic acid. In particular examples, the selected uniquely specific segments are noncontiguous, for example, separated by about 1500-2500 bp in the genomic target nucleic acid.

The segments of the selected genomic target nucleic acid sequence or other nucleic acid sequence are optionally screened for G/C nucleotide content (for example, percentage of bases in a nucleic acid sequence that are either guanine or cytosine). In some examples, the selected segments included in the probe hybridize to the genomic target nucleic acid under similar hybridization conditions. In addition to potentially maintaining more homogeneous probe fragment-target hybridization, probe G/C content below 65% can facilitate chemical synthesis of the DNA. Therefore, segments having a G/C nucleotide content of more than about 65% or less than about 30% (such as more than about 70% or 80% or less than about 30%, such as less than about 20% or 15%) may be removed. Methods for determining G/C nucleotide content of a sequence are known in the art. In some examples, G/C content may be calculated using the formula [(G+C)/(A+T+G+C)]×100. In other examples, methods for determining G/C content include a computer implemented algorithm, such as OligoCalc (Kibbe, Nucl. Acids Res. 35:W43-46, 2007; available on the World Wide Web at basic.northwestern.edu/biotools/oligocalc.html) or a macro-embedded spreadsheet file. In another example, the MATLAB® programming language can be used to analyze the percent G/C content of a sequence.

The segments of the selected genomic target nucleic acid sequence or other nucleic acid sequence are optionally screened for endonuclease restriction sites (such as type II restriction sites, for example, AscI/PacI, BbsI, BsmBI, BsaI, BtgZI, AarI, and SapI). Presence of such sequences can make gene synthesis and/or subsequent subcloning difficult, and eliminating such sequences creates a wider variety of DNA cloning options. Therefore, in some examples, segments including one or more type II restriction sites selected from AscI/PacI, BbsI, BsmBI, BsaI, BtgZI, AarI, and SapI are removed. Methods for determining the presence of restriction sites are known in the art. In some examples, methods for identifying restriction enzyme sites include a computer implemented algorithm, such as NEBcutter (New England BioLabs, Ipswich, Mass.; available on the internet at tools.neb.com/NEBcutter2/index.php) or Sequencher® (Gene Codes Corp., Ann Arbor, Mich.). In other examples, methods for identifying restriction sites utilize the MATLAB® programming language and software.

A skilled artisan will appreciate that hybridization between a probe and that of a target sequence depends on a number of factors, regardless of whether the probe is a probe produced using previously known methods (such as a “repeat-free” probe) or a uniquely specific probe of the present disclosure. For example, homology between a nucleic acid probe and its target sequence is important in hybridization kinetics, as are hybridization conditions, which can vary according to individual applications. For example, the stringency of hybridization conditions, washes, etc., such as those typically employed during microarray analysis may require different G/C content to preserve probe/target hybridizations than, for example, hybridization conditions typically utilized for in situ hybridization on tissue samples. As such, the G/C content of a probe useful in maintaining probe/target hybridizations may vary from application to application. For example, if the probe is intended for use in microarray applications, segments having a G/C nucleotide content of more than about 60% or less than about 30% (such as more than about 65%, 70%, or 80% or less than about 30%, such as less than about 20% or 15%) may be removed. In other examples, segments having a G/C nucleotide content of more than about 50% (such as more than about 55%, 60%, or 65%) are removed for probes intended for use in microarray applications.

1. In silico Identification of Uniquely Specific Segments

In some embodiments, following selection of genomic target nucleic acid sequence, optional repeat masking, separation into segments of the selected length, and optional screening for G/C nucleotide content and/or presence of selected restriction sites, individual segments (such as 100 base pair segments) are screened in silico to identify segments which have a sequence that is uniquely specific (such as represented only once in the genome of the organism). Segments that are uniquely specific are selected as binding regions, which are then joined (for example, ligated or linked) to produce the desired uniquely specific nucleic acid probe.

In other embodiments, following selection of a nucleic acid sequence from a genome of an organism divergent from the target genome of interest, optional repeat masking, separation into segments of the selected length, and optional screening for G/C nucleotide content and/or presence of selected restriction sites, individual segments (such as 100 base pair segments) are screened in silico to identify segments which are not represented in a target genome (such as a genome from an organism other than the starting nucleic acid sequence). Segments which are not represented in the genome of interest (for example, having no sequence identity to the target genome) are selected and may be synthesized for further testing or use (for example as a negative control on a probe array or in DNA-based signal amplification).

In some examples, each segment is compared to the genomic nucleic acid sequence of the organism from which the genomic target nucleic acid sequence is selected. In other examples, each segment is compared to the genomic nucleic acid sequence of the target genome (such as a genome other than the genome including the selected nucleic acid sequence of interest). Homology (for example, sequence identity) with the target nucleic acid sequence, as well as any non-target nucleic acid sequence in the genome is identified (for example, displayed as a sequence alignment). In a particular example, homology with the genome of the organism is identified and displayed using the computer algorithm BLAT (Blast-Like Analysis Tool; Kent, Genome Res. 12:656-644, 2002).

BLAT is an alignment tool which compares an input sequence to an index derived from an entire genome assembly. DNA BLAT keeps an index consisting of all non-overlapping 11-mers of an entire genome in random access memory, except for those areas that include high levels of repetitive sequence. BLAT scans through the input sequence to find areas of probable homology, which are then loaded into memory for a detailed alignment. DNA BLAT is designed to find sequences of 95% and greater similarity of length 25 bases or more. It may miss more divergent or shorter sequence alignments; however, BLAT will find perfect sequence matches of as few as 20-25 bases. In some examples, any segments including a perfect sequence match of more than about 20 bp (such as 20, 21, 22, 23, 24, 25 bp, or more) are eliminated.

In contrast, BLAST is an alignment tool which compares an input sequence to a database of GenBank sequences (Altschul et al., J. Mol. Biol. 215:403-410, 1990; Altschul et al., Nucl. Acids Res. 25:3389-3402, 1997). BLAST builds an index from the input sequence and scans linearly through the database. BLAST is less sensitive than BLAT for detecting uniquely specific nucleic acid sequences in a genomic target nucleic acid sequence. Due to the algorithm used in BLAST, sensitivity is sacrificed for speed, thus BLAST determines “best fit” and will not generate uniquely specific nucleic acid sequences. For example, BLAST will produce false positives (for example, identify a sequence segment as occurring only one time in the genome, where BLAT will identify multiple areas of homology in the genome to the same sequence segment). Therefore, BLAST is generally not suitable for use in the methods described herein.

The acceptance criterion for including a segment in a uniquely specific probe is a segment that is complementary to a uniquely specific nucleic acid sequence, such as a segment that is homologous to one and only one region of the genome (for example, the genomic target nucleic acid molecule). An accepted segment (designated a “binding region” or a “uniquely specific binding region”) may be included in a nucleic acid probe produced by the methods disclosed herein. Any segment that has homology (for example, is identical to another sequence over at least about 20-25 consecutive bp) to more than one region of the genome fails the acceptance criterion, and is not included in the nucleic acid probe. If a probe target area does not yield enough uniquely specific nucleic acid sequences, it can be supplemented with nucleic acid segments that include some nucleotides (for example, about 25 or less) that are identical to more than one region (such as 10 or less, for example, 2, 3, 4, 5, 6, 7, 8, 9, or 10 regions) of the genome may be included in the probe.

In one example, the acceptance criteria for a segment that is not represented in the target genome is a segment that does not return a positive result when compared to the target genome (for example, utilizing in silico methods, such as BLAT).

Uniquely specific binding regions and/or uniquely distinct nucleic acid molecules selected using the in silico methods described above may optionally be tested empirically for the presence or absence of hybridization with genomic DNA (for example from the target genome). In some examples, the testing identifies the presence of repetitive or other non-unique sequences (such as previously unidentified repetitive sequences) in the selected segments. In some examples, the selected segments (for example, binding regions or nucleic acids not represented in a genome of interest) are prepared (for example by oligonucleotide synthesis) and tested for hybridization with genomic DNA from the organism containing the genomic target nucleic acid (in the case of uniquely specific binding regions) or with genomic DNA from the target genome (in the case of a nucleic acid not represented in the target genome). Hybridization methods are well known in the art, such as membrane-based hybridization techniques (for example, Southern blot, slot-blot, or dot-blot). In a particular example, hybridization is tested by dot-blotting. For example, the sequence segments can be synthesized as oligonucleotides, spotted onto a membrane, and hybridized with labeled genomic DNA probe. In some example, if there is no hybridization (for example, no detectable hybridization) to the genomic DNA probe, the segment is confirmed to be a uniquely specific binding region and may be selected for inclusion in a nucleic acid probe produced by the methods disclosed herein. If there is any hybridization (for example, any detectable hybridization) to the genomic DNA probe, the segment may be excluded from the nucleic acid probe. In other examples, if there is no hybridization (for example, no detectable hybridization) to the genomic DNA probe, the segment may be selected as a segment that is not represented in the target genome. If there is any hybridization (for example, any detectable hybridization) to the genomic DNA probe, the segment may be identified as a segment that is represented in the target genome.

In other examples, a microarray including the selected segments (such as binding regions or segments not represented in the target genome) is prepared. In some examples, the array optionally includes positive and negative controls. Positive controls can include repetitive element sequences, similar to the examples given above, for example AluI alpha satellite (such as D17Z1), LINE element (such as Sau3), and/or telomeric sequences (such as pHuR93Telo). Negative controls can include genomic sequences from an unrelated organism (such as rice), or randomized sequences (such as those commonly used on commercially available arrays). In some examples, the microarray is probed with labeled total genomic DNA, such as DNA from the target genome. In other examples, the microarray is probed with labeled total genomic DNA (such as human total genomic DNA) and labeled repetitive DNA (such as Cot-1™ DNA). In some examples, the array is probed simultaneously with the total genomic DNA and the repetitive DNA. In other examples, two separate, identical, arrays are probed, one with the total genomic DNA and one with the repetitive DNA. Data is collected and analyzed by standard methods and software (for example, NimbleScan software, Roche Nimblegen).

In some examples, selection criteria are established to screen the test sequences (segments, tag sequences, or binding regions) by deriving a linear regression of all the positive control sequences and decreasing the linear regression by one standard deviation. In addition, the minimum human genomic score from the positive controls (such as the AluI positive controls), and a predetermined value (such as 12) for the repetitive DNA probe (such as Cot-1™) are established as additional positive control cutoffs. The cutoff for negative controls is established by using the mean of the total genomic DNA score of the negative control sequences. Such cutoffs differentiate the hybridization intensities of a subset of test sequences, such that the sequences that perform more similar to the positive and negative controls are segregated. Sequences that fall within the selection criteria are included in the probe, whereas sequences that fall outside of the selection criteria are eliminated. In some examples, sequences that fall within the selection criteria are considered to be uniquely specific sequences (such as sequences that occur only once in the genome of the organism). In other examples, sequences that fall within the selection criteria are considered to be sequences not represented in the target genome (such as sequences with no sequence identity to the target genome). One skilled in the art of array data analysis will understand that many different statistical methods can be used to derive meaningful cutoffs that can be used to exclude/include test sequences.

2. Empiric Identification of Uniquely Specific Segments

In other embodiments, empiric testing of enumerated sequence is utilized to identify uniquely specific binding regions. Empiric analysis may be used in place of in silico methods (for example, BLAT analysis), described in section 1 (above).

In some examples, following selection of genomic target nucleic acid sequence, optional repeat masking, separation into segments of the selected length, and optional screening for G/C nucleotide content and/or presence of selected restriction sites, individual segments (such as 15-500 base pair segments, for example, 100 base pair segments) are synthesized and attached to an array. Any number of individual segments for testing (such as at least 10, 50, 100, 200, 300, 400, 500, 600, 700, 800, 900, 1000, 2000, 4000, 5000, 8000, 10,000, 50,000, 100,000, 200,000, or more) can be attached to the array. In some examples, the array optionally includes positive and negative controls. Positive controls can include repetitive element sequences, for example AluI alpha satellite (such as D17Z1), LINE element (such as Sau3), and/or telomeric sequences (such as pHuR93Telo). In particular examples, a positive control is a sequence with a known copy number in the genome of the organism including the target genomic sequence. In some examples, a negative control is a randomized sequence, such as a sequence that has little to no homology to the genome of the organism. Negative controls can also include genomic sequences from an unrelated organism, such as from a plant (for example, rice), bacterial, viral, or yeast genome.

The arrays of the present disclosure can be prepared by a variety of approaches. In one example, nucleic acid molecules are synthesized separately and then attached to a solid support (see U.S. Pat. No. 6,013,789). In another example, nucleic acid molecules are synthesized directly onto the support to provide the desired array (see U.S. Pat. No. 5,554,501). Suitable methods for covalently coupling nucleic acids to a solid support and for directly synthesizing the nucleic acids onto the support are known to those working in the field; a summary of suitable methods can be found in Matson et al., Anal. Biochem. 217:306-10, 1994. In one example, the nucleic acid molecules are synthesized onto the support using conventional chemical techniques for preparing oligonucleotides on solid supports (such as PCT applications WO 85/01051 and WO 89/10977, or U.S. Pat. No. 5,554,501). The solid support of the array can be formed from an organic polymer. Suitable materials for the solid support include, but are not limited to: polypropylene, polyethylene, polybutylene, polyisobutylene, polybutadiene, polyisoprene, polyvinylpyrrolidine, polytetrafluoroethylene, polyvinylidene difluoride, polyfluoroethylene-propylene, polyethylenevinyl alcohol, polymethylpentene, polycholorotrifluoroethylene, polysulformes, hydroxylated biaxially oriented polypropylene, aminated biaxially oriented polypropylene, thiolated biaxially oriented polypropylene, ethyleneacrylic acid, thylene methacrylic acid, and blends of copolymers thereof (see U.S. Pat. No. 5,985,567).

In some examples, the microarray is probed with labeled total genomic DNA from the organism of interest and labeled repetitive DNA from the genome of the organism. In a particular example, human total genomic DNA and Cot-1™ DNA are used. In some examples, the array is probed sequentially with the total genomic DNA and the repetitive DNA. In other examples, two separate, identical, arrays are probed, one with the total genomic DNA and one with the repetitive DNA. Data is collected and analyzed by standard methods and software (for example, NimbleScan software, Roche Nimblegen).

In some examples, uniquely specific sequences are selected by deriving a linear regression of hybridization scores of total genomic DNA and blocking DNA and selecting sequences falling within one or more predetermined cutoffs. In some examples, selection criteria are established to screen the test sequences by deriving a linear regression of all the positive control sequences and decreasing the linear regression by one standard deviation. In addition, the minimum human genomic score from a positive control (such as an AluI positive control), and a predetermined value (such as 11, 12, 13, or 14, for example, 12) for the blocking DNA (such as the Cot-1™ DNA) are established as additional positive control cutoffs. The cutoff for negative controls can be established by using the mean of the total human genomic DNA score of the negative control sequences. Such cutoffs differentiate the hybridization intensities of a subset of test sequences, such that the sequences that perform more similarly to the positive and negative controls will be segregated. Sequences that fall within the selection criteria are included in the probe, whereas sequences that fall outside of the selection criteria are eliminated. In some examples, sequences that fall within the selection criteria are considered to be uniquely specific sequences (such as sequences that occur only once in the genome of the organism). One skilled in the art of array data analysis will understand that many different statistical methods can be used to derive meaningful cutoffs that can be used to exclude/include test sequences. In further examples, if the array does not include positive and negative controls, the sequence selection criteria is the distance from the population origin of the mean of all sequences included in the array. In this case, a defined number of sequences are chosen with respect to their radial distance from this origin, which can be established hierarchically.

In some embodiments, the uniquely specific sequences selected using the criteria described above are placed in an order and orientation that is as they occur in the genomic target. In other examples, the methods of determining an order and orientation of the selected sequences in the probe can include those methods described in Part IV, Section B (below).

B. Determining Order and Orientation of Uniquely Specific Sequences

In some examples, the disclosed methods further include determining an order and orientation of the selected binding regions complementary to uniquely specific nucleic acid sequences, prior to joining the binding regions to generate the nucleic acid probe (identifying a pre-determined order and orientation). The uniquely specific binding regions are selected as described in Section IV, Part A (above). However, it is possible that non-uniquely specific nucleic acid sequence (such as a nucleic acid sequence that is represented more than once in the haploid genome, for example, a repetitive sequence or homology to a non-target nucleic acid) may be generated when the selected uniquely specific binding regions are joined. For example, a non-uniquely specific sequence may be generated from a sequence that includes an overlapping region between two or more binding regions (such as at the site where two uniquely specific sequences are joined). Therefore, the nucleic acid probe sequence can be analyzed to assure that the generated probe does not include non-uniquely specific nucleic acid sequences. If the probe contains non-uniquely specific nucleic acid sequence, the order and/or orientation of the binding regions in the probe is changed and re-analyzed.

Determining the order and orientation of the binding regions in the probe includes placing the selected uniquely specific binding regions in an initial order and orientation. In some examples, the binding regions utilized to produce that initial order include a number of uniquely specific binding regions that provide a convenient total sequence length. The total sequence length can include any length that can be included in a vector (such as a plasmid, cosmid, bacterial artificial chromosome or yeast artificial chromosome), including, but not limited to at least 1000 bp, at least 10,000 bp, at least 20,000 bp, at least 50,000 bp, for example about 1000 bp to about 60,000 bp (for example, about 1000 bp, 2000 bp, 3000 bp, 4000 bp, 4500 bp, 5000 bp, 5500 bp, 6000 bp, 7000 bp, 8000 bp, 10,000 bp, 20,000 bp, 30,000 bp, 40,000 bp, 50,000 bp, or 60,000 bp) total length of uniquely specific binding regions. In some examples, the total size of the selected uniquely specific binding regions from a genomic target nucleic acid sequence may exceed a sequence length that may be conveniently included in a plasmid vector. In such examples, the selected uniquely specific binding regions may be divided into groups, such that each group includes a total sequence length suitable for insertion in a vector (such as a plasmid, cosmid, bacterial artificial chromosome or yeast artificial chromosome).

In some examples, the initial ordering of the selected uniquely specific binding regions may be in the order that the uniquely specific binding regions occur in the genomic target nucleic acid. For example, the selected binding region that is located most 5′ in the genomic target nucleic acid is placed first in the initial ordering, followed by the selected binding region that occurs next in the genomic target nucleic acid moving in a 5′ to 3′ direction, and so on, until the selected binding region that is located most 3′ in the genomic target nucleic acid is placed last in the initial ordering. In addition, each of the binding regions is placed in the same orientation in the initial ordering as it occurs in the genomic target nucleic acid. Alternatively, each of the binding regions may be placed in reverse orientation in the initial ordering as it occurs in the genomic target nucleic acid, or a mixture of forward and reverse orientations may be used.

In another example, the initial ordering of the selected uniquely specific binding regions may be every 1+n binding regions as they occur in the genomic target nucleic acid, where n is 1, 2, 3, 4, 5, 6, 7, 8, 9, or 10. For example, the initial ordering could be every second selected binding region, every third selected binding region, every fourth selected binding region, every fifth selected binding region, and so on. The initial ordering of the selected uniquely specific binding regions may also include the reverse order to the order that they occur in the genomic target nucleic acid. The orientation of the selected uniquely specific binding regions may be in the orientation that they occur in the genomic target nucleic acid, the reverse orientation, or may be random. In other examples, the initial ordering of the selected uniquely specific binding regions may be in reverse order from how they occur in the genome, or may be in a randomly selected order.

Following the initial ordering of the binding regions, the resulting sequence is analyzed for the de novo generation of any non-uniquely specific nucleic acid sequence. This is performed as described for the selection of uniquely specific segments (Section IV, Part A, above). In some examples, the initial order and orientation of the binding regions does not include any non-uniquely specific nucleic acid sequences. In such an example, the initial ordering is the same order and orientation selected for linking the binding regions to generate the probe (the “pre-determined” order and orientation).

In other examples, the initial order and orientation of the binding regions generates at least one non-uniquely specific segment. If the initial ordering generates at least one non-uniquely specific segment, the order and orientation of the selected binding regions is adjusted to identify an order and orientation that consists of uniquely specific nucleic acid sequences. In one example, the binding region that resulted in the formation of a non-uniquely specific nucleic acid sequence in the initial ordering is moved to an end of the ordered binding regions (for example, the 5′ end or the 3′ end of the ordered binding regions).

In other examples, the binding region that resulted in the formation of a non-uniquely specific nucleic acid sequence may remain in the same order, but be placed in the opposite orientation, or it may be both moved to an end of the ordered binding region and placed in the opposite orientation. In another example, the binding region that resulted in the formation of a non-uniquely specific nucleic acid sequence may be excluded from the probe. In a further example, all of the selected binding regions may be re-ordered, for example by choosing a different order and/or orientation, such as those described above for the initial ordering. The sequence consisting of the adjusted or re-ordered segments is then analyzed for the de novo generation of any non-uniquely specific nucleic acid sequence. This is performed as described for the selection of uniquely specific segments (Section IV, Part A, above).

In some examples, the adjusted order and orientation of the binding regions does not include any non-uniquely specific nucleic acid sequences. In such an example, the adjusted order and orientation is the order and orientation selected for joining the binding regions to generate the probe (the “pre-determined” order and orientation). In other examples, the adjusted ordering generates at least one non-uniquely specific segment. If the adjusted ordering generates at least one non-uniquely specific segment, the order and orientation of the selected binding regions is re-adjusted to identify an order and orientation that consists of uniquely specific nucleic acid sequences, as described above. This process is repeated as many times as necessary to identify an order and orientation of the selected binding regions that does not include any non-uniquely specific nucleic acid sequences.

Once an order and orientation of the uniquely specific binding regions is determined, the binding regions are joined (e.g., ligated or linked) in the pre-determined order and orientation. In some examples, the individual binding region sequences are produced (for example by oligonucleotide synthesis or by amplification of the sequences from the genomic target nucleic acid) and joined together in the selected order and orientation. In other examples, the nucleic acid probe is synthesized as a series of oligonucleotides (such as individual oligonucleotides of about 20-500 bp), which are joined together. For example, the binding regions may be joined or ligated to one another enzymatically (e.g., using a ligase). For example, binding regions can be joined in a blunt-end ligation or at a restriction site. In another example, the binding regions may be synthesized with complementary nucleic acid overhangs (such as at least a 3 bp overhang), annealed, and joined to one another, for example with a ligase. Chemical ligation and amplification can also be used to join binding regions. In some examples, the binding regions are separated by linkers. In another example, the entire nucleic acid probe including the selected binding regions in the selected order and orientation is synthesized and the binding regions are directly joined during synthesis. In particular examples, the plurality of joined (e.g., ligated or linked) binding regions are inserted into a plasmid vector to allow production of the nucleic acid probe by standard molecular biology techniques.

### V. Target Nucleic Acid Sequences

In some examples, target nucleic acid sequences or molecules include genomic DNA target sequences. Nucleic acid molecules including at least a first binding region and a second binding region complementary to uniquely specific nucleic acid sequences can be generated which correspond to essentially any genomic target sequence. In some examples, a target sequence is selected that is associated with a disease or condition, such that detection of hybridization can be used to infer information (such as diagnostic or prognostic information for the subject from whom the sample is obtained) relating to the disease or condition. In a specific example, the genomic target nucleic acid sequence is selected from a target genome such as a eukaryotic genome, for example, a mammalian genome, such as a human genome.

The disclosed uniquely specific nucleic acid molecules can be generated which correspond to essentially any genomic target sequence that includes at least a portion of uniquely specific DNA. For example, the genomic target sequence can be a portion of a eukaryotic genome, such as a mammalian (e.g., human) genome. The uniquely specific nucleic acid molecules and probes including such molecules can correspond to one or more individual genes (including coding and/or non-coding portions of genes), regions of one or more chromosomes (e.g., a region that includes one or more genes of interest or includes no known genes) or even one or more entire chromosomes.

In some embodiments, a target nucleic acid sequence or molecule includes any nucleic acid sequence or molecule of interest. Nucleic acid molecules that are not represented in a target genome can be generated for essentially any target genome of interest, such as one that is divergent from the target genome. In some examples, the genome of interest is a mammalian genome (such as a eukaryotic genome or human genome), and the divergent genome is from a plant genome, such as an Oryza genome (for example an Oryza sativa genome), an Arabidopsis genome (for example, an Arabidopsis thaliana genome), or an insect genome, such as a Drosophila melanogaster genome.

The target nucleic acid sequence (e.g., genomic target nucleic acid sequence or other genomic nucleic acid sequence of interest, such as a genome divergent from the target) can span any number of base pairs. In one example, such as a genomic target nucleic acid sequence selected from a mammalian or other genome with substantial interspersed repetitive nucleic acid sequence (for example, a human genome), the target nucleic acid sequence spans at least 100,000 bp. In specific examples, a target nucleic acid sequence (e.g., genomic target nucleic acid sequence) is at least about 100,000 bp, such as at least about 150,000, 250,000, 500,000, 600,000, 700,000, 800,000, 900,000, 1,000,000, 1,500,000, 2,000,000, 3,000,000, 4,000,000 bp, or more (such as an entire chromosome).). In further examples, the genomic nucleic acid sequence includes an entire genome (such as an Oryza genome, an Arabidopsis genome, or a Drosophila genome) or a portion thereof, such as about one tenth, about one quarter, about one third, about half, about two thirds, or about three quarters, or more of a genome sequence.

In specific non-limiting examples, a genomic target nucleic acid sequence associated with a neoplasm (for example, a cancer) is selected. Numerous chromosome abnormalities (including translocations and other rearrangements, reduplication (amplification) or deletion) have been identified in neoplastic cells, especially in cancer cells, such as B cell and T cell leukemias, lymphomas, breast cancer, colon cancer, neurological cancers and the like. Therefore, in some examples, at least a portion of the target nucleic acid sequence (e.g., genomic target nucleic acid sequence) is reduplicated or deleted in at least a subset of cells in a sample.

Translocations involving oncogenes are known for several human malignancies. For example, chromosomal rearrangements involving the SYT gene located in the breakpoint region of chromosome 18q11.2 are common among synovial sarcoma soft tissue tumors. The t(18q11.2) translocation can be identified, for example, using probes with different labels: the first probe includes uniquely specific nucleic acid molecules generated from a target nucleic acid sequence that extends distally from the SYT gene, and the second probe includes uniquely specific nucleic acid molecules generated from a target nucleic acid sequence that extends 3′ or proximal to the SYT gene. When probes corresponding to these target nucleic acid sequences (e.g., genomic target nucleic acid sequences) are used in an in situ hybridization procedure, normal cells, which lack a t(18q11.2) in the SYT gene region, exhibit two fusion (generated by the two labels in close proximity) signals, reflecting the two intact copies of SYT. Abnormal cells with a t(18q11.2) exhibit a single fusion signal.

Numerous examples of reduplication of genes (also known as gene amplification) involved in neoplastic transformation have been observed, and can be detected cytogenetically by in situ hybridization using the disclosed probes. In one example, the genomic target nucleic acid sequence is selected to include a gene (e.g., an oncogene) that is reduplicated in one or more malignancies (e.g., a human malignancy). For example, HER2, also known as c-erbB2 or HER2/neu, is a gene that plays a role in the regulation of cell growth (a representative human HER2 genomic sequence is provided at GENBANK™ Accession No. NC_{—}000017, nucleotides 35097919-35138441). The gene codes for a 185 kD transmembrane cell surface receptor that is a member of the tyrosine kinase family. HER2 is amplified in human breast, ovarian, gastric, and other cancers. Therefore, a HER2 gene (or a region of chromosome 17 that includes a HER2 gene) can be used as a genomic target nucleic acid sequence to generate probes that include uniquely specific binding regions for HER2.

In other examples, a genomic target nucleic acid sequence is selected that is a tumor suppressor gene that is deleted (lost) in malignant cells. For example, the p16 region (including D9S1749, D9S1747, p16(INK4A), p14(ARF), D9S1748, p15(INK4B), and D9S1752) located on chromosome 9p21 is deleted in certain bladder cancers. Chromosomal deletions involving the distal region of the short arm of chromosome 1 (that encompasses, for example, SHGC57243, TP73, EGFL3, ABL2, ANGPTL1, and SHGC-1322), and the pericentromeric region (e.g., 19p13-19g13) of chromosome 19 (that encompasses, for example, MAN2B1, ZNF443, ZNF44, CRX, GLTSCR2, and GLTSCR1)) are characteristic molecular features of certain types of solid tumors of the central nervous system.

The aforementioned examples are provided solely for purpose of illustration and are not intended to be limiting. Numerous other cytogenetic abnormalities that correlate with neoplastic transformation and/or growth are known to those of skill in the art. Genomic target nucleic acid sequences, which have been correlated with neoplastic transformation and which are useful in the disclosed methods and for which disclosed probes can be prepared, also include the EGFR gene (7p12; e.g., GENBANK™ Accession No. NC_{—}000007, nucleotides 55054219-55242525), the MET gene (7q31; e.g., GENBANK™ Accession No. NC_{—}000007, nucleotides 116099695-116225676), the C-MYC gene (8q24.21; e.g., GENBANK™ Accession No. NC_{—}000008, nucleotides 128817498-128822856), IGF1R (15q26.3; e.g., GENBANK™ Accession No. NC_{—}000015, nucleotides 97010284-97325282), D5S271 (5p15.2), KRAS (12p12.1; e.g. GENBANK™ Accession No. NC_{—}000012, complement, nucleotides 25249447-25295121), TYMS (18p11.32; e.g., GENBANK™ Accession No. NC_{—}000018, nucleotides 647651-663492), CDK4 (12q14; e.g., GENBANK™ Accession No. NC_{—}000012, nucleotides 58142003-58146164, complement), CCND1 (11q13, GENBANK™ Accession No. NC_{—}000011, nucleotides 69455873-69469242), MYB (6q22-q23, GENBANK™ Accession No. NC_{—}000006, nucleotides 135502453-135540311), lipoprotein lipase (LPL) gene (8p22; e.g., GENBANK™ Accession No. NC_{—}000008, nucleotides 19840862-19869050), RB1 (13q14; e.g., GENBANK™ Accession No. NC_{—}000013, nucleotides 47775884-47954027), p53 (17p13.1; e.g., GENBANK™ Accession No. NC_{—}000017, complement, nucleotides 7512445-7531642), N-MYC (2p24; e.g., GENBANK™ Accession No. NC_{—}000002, complement, nucleotides 15998134-16004580), CHOP (12q13; e.g., GENBANK™ Accession No. NC_{—}000012, complement, nucleotides 56196638-56200567), FUS (16p11.2; e.g., GENBANK™ Accession No. NC_{—}000016, nucleotides 31098954-31110601), FKHR (13p14; e.g., GENBANK™ Accession No. NC_{—}000013, complement, nucleotides 40027817-40138734), as well as, for example: ALK (2p23; e.g., GENBANK™ Accession No. NC_{—}000002, complement, nucleotides 29269144-29997936), Ig heavy chain, CCND1 (11q13; e.g., GENBANK™ Accession No. NC_{—}000011, nucleotides 69165054-69178423), BCL2 (18q21.3; e.g., GENBANK™ Accession No. NC_{—}000018, complement, nucleotides 58941559-59137593), BCL6 (3q27; e.g., GENBANK™ Accession No. NC_{—}000003, complement, nucleotides 188921859-188946169), AP1 (1p32-p31; e.g., GENBANK™ Accession No. NC_{—}000001, complement, nucleotides 59019051-59022373), TOP2A (17q21-q22; e.g., GENBANK™ Accession No. NC_{—}000017, complement, nucleotides 35798321-35827695), TMPRSS (21q22.3; e.g., GENBANK™ Accession No. NC_{—}000021, complement, nucleotides 41758351-41801948), ERG (21q22.3; e.g., GENBANK™ Accession No. NC_{—}000021, complement, nucleotides 38675671-38955488); ETV1 (7p21.3; e.g., GENBANK™ Accession No. NC_{—}000007, complement, nucleotides 13897379-13995289), EWS (22q12.2; e.g., GENBANK™ Accession No. NC_{—}000022, nucleotides 27994017-28026515); FLI1 (11q24.1-q24.3; e.g., GENBANK™ Accession No. NC_{—}000011, nucleotides 128069199-128187521), PAX3 (2q35-q37; e.g., GENBANK™ Accession No. NC_{—}000002, complement, nucleotides 222772851-222871944), PAX7 (1p36.2-p36.12; e.g., GENBANK™ Accession No. NC_{—}000001, nucleotides 18830087-18935219), PTEN (10q23.3; e.g., GENBANK™ Accession No. NC_{—}000010, nucleotides 89613175-89718512), AKT2 (19q13.1-q13.2; e.g., GENBANK™ Accession No. NC_{—}000019, complement, nucleotides 45428064-45483105), MYCL1 (1p34.2; e.g., GENBANK™ Accession No. NC_{—}000001, complement, nucleotides 40133685-40140274), REL (2p13-p12; e.g., GENBANK™ Accession No. NC_{—}000002, nucleotides 60962256-61003682) and CSF1R (5q33-q35; e.g., GENBANK™ Accession No. NC_{—}000005, complement, nucleotides 149413051-149473128). A disclosed probe or method may include a region of the respective human chromosome containing at least a portion of any one (or more, as applicable) of the foregoing genes.

In certain embodiments, the probe specific for the genomic target nucleic acid molecule is assayed (in the same or a different but analogous sample) in combination with a second probe that provides an indication of chromosome number, such as a chromosome specific (e.g., centromere) probe. For example, a probe specific for a region of chromosome 17 containing at least uniquely specific nucleic acid sequences of the HER2 gene (a HER2 probe) can be used in combination with a CEP 17 probe that hybridizes to the alpha satellite DNA located at the centromere of chromosome 17 (17p11.1-q11.1). Inclusion of the CEP 17 probe allows for the relative copy number of the HER2 gene to be determined. For example, normal samples will have a HER2/CEP17 ratio of less than 2, whereas samples in which the HER2 gene is reduplicated will have a HER2/CEP17 ratio of greater than 2.0. Similarly, CEP centromere probes corresponding to the location of any other selected genomic target sequence can also be used in combination with a probe for a unique target on the same (or a different) chromosome.

### VI. Detectable Labels and Methods of Labeling

In some examples, the nucleic acid probes or nucleic acid tags generated by the disclosed methods can include one or more labels, for example to permit detection of a target nucleic acid molecule using the disclosed probes or tags. In various applications, such as in situ hybridization procedures, a nucleic acid probe or tag includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. The disclosure is not limited to the use of particular labels, although examples are provided.

A label associated with one or more nucleic acid molecules (such as a probe or tag generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook—A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl)aminonaphthalene-1-sulfonic acid (EDANS),4-amino-N-[3-vinylsulfonyl)phenyl]naphthalimide-3,5 disulfonate (Lucifer Yellow VS), N-(4-anilino-1-naphthyl)maleimide, anthranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate (FITC), and QFITC(XRITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives.

Other suitable fluorophores include thiol-reactive europium chelates which emit at approximately 617 nm (Heyduk and Heyduk, Analyt. Biochem. 248:216-27, 1997; J. Biol. Chem. 274:3315-22, 1999), as well as GFP, Lissamine™ diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6,130,101 and 6, 716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682596; and 6,649,138). Semiconductor nanocrystals are microscopic particles having size-dependent optical and/or electrical properties. When semiconductor nanocrystals are illuminated with a primary energy source, a secondary emission of energy occurs of a frequency that corresponds to the bandgap of the semiconductor material used in the semiconductor nanocrystal. This emission can be detected as colored light of a specific wavelength or fluorescence. Semiconductor nanocrystals with different spectral characteristics are described in e.g., U.S. Pat. No. 6,602,671. Semiconductor nanocrystals that can be coupled to a variety of biological molecules (including dNTPs and/or nucleic acids) or substrates by techniques described in, for example, Bruchez et al., Science 281:2013-2016, 1998; Chan et al., Science 281:2016-2018, 1998; and U.S. Pat. No. 6,274,323.

Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (published May 27, 1999). Separate populations of semiconductor nanocrystals can be produced that are identifiable based on their different spectral characteristics. For example, semiconductor nanocrystals can be produced that emit light of different colors based on their composition, size or size and composition. For example, quantum dots that emit light at different wavelengths based on size (565 nm, 655 nm, 705 nm, or 800 nm emission wavelengths), which are suitable as fluorescent labels in the probes disclosed herein are available from Life Technologies (Carlsbad, Calif.).

Additional labels include, for example, radioisotopes (such as ³H), metal chelates such as DOTA and DPTA chelates of radioactive or paramagnetic metal ions like Gd³⁺, and liposomes.

Detectable labels that can be used with nucleic acid molecules (such as a probe or tag generated by the disclosed methods) also include enzymes, for example horseradish peroxidase, alkaline phosphatase, acid phosphatase, glucose oxidase, β-galactosidase, β-glucuronidase, or β-lactamase. Where the detectable label includes an enzyme, a chromogen, fluorogenic compound, or luminogenic compound can be used in combination with the enzyme to generate a detectable signal (numerous of such compounds are commercially available, for example, from Life Technologies, Carlsbad, Calif.). Particular examples of chromogenic compounds include diaminobenzidine (DAB), 4-nitrophenylphosphate (pNPP), fast red, fast blue, bromochloroindolyl phosphate (BCIP), nitro blue tetrazolium (NBT), BCIP/NBT, AP Orange, AP blue, tetramethylbenzidine (TMB), 2,2′-azino-di-[3-ethylbenzothiazoline sulphonate] (ABTS), o-dianisidine, 4-chloronaphthol (4-CN), nitrophenyl-β-D-galactopyranoside (ONPG), o-phenylenediamine (OPD), 5-bromo-4-chloro-3-indolyl-β-galactopyranoside (X-Gal), methylumbelliferyl-β-D-galactopyranoside (MU-Gal), p-nitrophenyl-α-D-galactopyranoside (PNP), 5-bromo-4-chloro-3-indolyl-β-D-glucuronide (X-Gluc), 3-amino-9-ethyl carbazol (AEC), fuchsin, iodonitrotetrazolium (INT), tetrazolium blue and tetrazolium violet.

Alternatively, an enzyme can be used in a metallographic detection scheme. For example, silver in situ hybridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redox-active agent reduces the metal ion, causing it to form a detectable precipitate. (See, for example, U.S. Patent Application Publication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

In non-limiting examples, nucleic acid probes or tags (such as a probe or tag generated by the disclosed methods) are labeled with dNTPs covalently attached to hapten molecules (such as a nitro-aromatic compound (e.g., dinitrophenyl (DNP)), biotin, fluorescein, digoxigenin, etc.). Methods for conjugating haptens and other labels to dNTPs (e.g., to facilitate incorporation into labeled probes) are well known in the art. For examples of procedures, see, e.g., U.S. Pat. Nos. 5,258,507, 4,772,691, 5,328,824, and 4,711,955. Indeed, numerous labeled dNTPs are available commercially, for example from Life Technologies (Molecular Probes, Eugene, Oreg.). A label can be directly or indirectly attached to a dNTP at any location on the dNTP, such as a phosphate (e.g., α, β or γ phosphate) or a sugar. Detection of labeled nucleic acid molecules can be accomplished by contacting the hapten-labeled nucleic acid molecules bound to the genomic target sequence with a primary anti-hapten antibody. In one example, the primary anti-hapten antibody (such as a mouse anti-hapten antibody) is directly labeled with an enzyme. In another example, a secondary anti-antibody (such as a goat anti-mouse IgG antibody) conjugated to an enzyme is used for signal amplification. In CISH a chromogenic substrate is added, for SISH, silver ions and other reagents as outlined in the referenced patents/applications are added.

In some examples, a probe is labeled by incorporating one or more labeled dNTPs using an enzymatic (polymerization) reaction. For example, the nucleic acid probe (such as at least two uniquely specific binding regions, such as incorporated into a plasmid vector) can be labeled by nick translation (using, for example, biotin, 2,4-dinitrophenol, digoxigenin, etc.) or by random primer extension with terminal transferase (e.g., 3′ end tailing). In some examples, the nucleic probe is labeled by a modified nick translation reaction where the ratio of DNA polymerase Ito deoxyribonuclease I (DNase I) is modified to produce greater than 100% of the starting material. In particular examples, the nick translation reaction includes DNA polymerase Ito DNase I at a ratio of at least about 800:1, such as at least 2000:1, at least 4000:1, at least 8000:1, at least 10,000:1, at least 12,000:1, at least 16,000:1, such as about 800:1 to 24,000:1 and the reaction is carried out overnight (for example, for about 16-22 hours) at a substantially isothermal temperature, for example, at about 16° C. to 25° C. (such as room temperature). See, e.g., U.S. Provisional Patent Application No. 61/291,741, entitled “Methods and Compositions for Nucleic Acid Labeling and Amplification,” filed on Dec. 31, 2009; incorporated herein by reference.

If the nucleic acid probe or tag includes multiple plasmids (such as 2, 3, 4, 5, 6, 7, 8, 9, 10, or more plasmids), the plasmids may be mixed in an equal molar ratio prior to performing the labeling reaction (such as nick translation or modified nick translation), to insure that all binding regions are equally abundant following labeling.

In other examples, chemical labeling procedures can also be employed. Numerous reagents (including hapten, fluorophore, and other labeled nucleotides) and other kits are commercially available for enzymatic labeling of nucleic acids, including nucleic acid probes produced by the methods disclosed herein. As will be apparent to those of skill in the art, any of the labels and detection procedures disclosed above are applicable in the context of labeling a probe, e.g., for use in in situ hybridization reactions. For example, the Amersham MULTIPRIME® DNA labeling system, various specific reagents and kits available from Molecular Probes/Life Technologies, or any other similar reagents or kits can be used to label the nucleic acids disclosed herein. In particular examples, the disclosed probes can be directly or indirectly labeled with a hapten, a ligand, a fluorescent moiety (e.g., a fluorophore or a semiconductor nanocrystal), a chromogenic moiety, or a radioisotope. For example, for indirect labeling, the label can be attached to nucleic acid molecules via a linker (e.g., PEG or biotin).

Additional methods that can be used to label nucleic acid molecules are provided in U.S. Application Pub. No. 2005/0158770.

### VII. Methods of Using Probes

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH). Exemplary uses are discussed below.

A. In Situ Hybridization

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The chromosome sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat anti-avidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). The enzyme reaction can be performed in, for example, AP buffer containing NBT/BCIP and stopped by incubation in 2×SSC. For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pinkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. J. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above, probes labeled with fluorophores (including fluorescent dyes and QUANTUM DOTS®) can be directly optically detected when performing FISH. Alternatively, the probe can be labeled with a non-fluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can in turn be labeled with a fluorophore. Optionally, the detectable label is attached directly to the antibody, receptor (or other specific binding agent). Alternatively, the detectable label is attached to the binding agent via a linker, such as a hydrazide thiol linker, a polyethylene glycol linker, or any other flexible attachment moiety with comparable reactivities. For example, a specific binding agent, such as an antibody, a receptor (or other anti-ligand), avidin, or the like can be covalently modified with a fluorophore (or other label) via a heterobifunctional polyalkyleneglycol linker such as a heterobifunctional polyethyleneglycol (PEG) linker. A heterobifunctional linker combines two different reactive groups selected, e.g., from a carbonyl-reactive group, an amine-reactive group, a thiol-reactive group and a photo-reactive group, the first of which attaches to the label and the second of which attaches to the specific binding agent.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/0117153.

In further examples, a signal amplification method is utilized, for example, to increase sensitivity of the probe. In particular examples, signal amplification is utilized with probes of about 5000 bp or less (such as about 5000, 4500, 4000, 3500, 3000, 2500, 2000, 1500, 1000, 900. 800, 700, 600, 500, 400, 300, 200, or 100 bp). One of skill in the art can select probes for which signal amplification is appropriate. For example, CAtalyzed Reporter Deposition (CARD), also known as Tyramide Signal Amplification (TSA™) may be utilized. In one variation of this method a biotinylated nucleic acid probe detects the presence of a target by binding thereto. Next a streptavidin-peroxidase conjugate is added. The streptavidin binds to the biotin. A substrate of biotinylated tyramide (tyramine is 4-(2-aminoethyl)phenol) is used, which presumably becomes a free radical when interacting with the peroxidase enzyme. The phenolic radical then reacts quickly with the surrounding material, thus depositing or fixing biotin in the vicinity. This process is repeated by providing more substrate (biotinylated tyramide) and building up more localized biotin. Finally, the “amplified” biotin deposit is detected with streptavidin attached to a fluorescent molecule. Alternatively, the amplified biotin deposit can be detected with avidin-peroxidase complex, that is then fed 3,3′-diaminobenzidine to produce a brown color. It has been found that tyramide attached to fluorescent molecules also serve as substrates for the enzyme, thus simplifying the procedure by eliminating steps.

In other examples, the signal amplification method utilizes branched DNA signal amplification. In some examples, target-specific oligonucleotides (label extenders and capture extenders) are hybridized with high stringency to the target nucleic acid. Capture extenders are designed to hybridize to the target and to capture probes, which are attached to a microwell plate. Label extenders are designed to hybridize to contiguous regions on the target and to provide sequences for hybridization of a preamplifier oligonucleotide. Signal amplification then begins with preamplifier probes hybridizing to label extenders. The preamplifier forms a stable hybrid only if it hybridizes to two adjacent label extenders. Other regions on the preamplifier are designed to hybridize to multiple bDNA amplifier molecules that create a branched structure. Finally, alkaline phosphatase (AP)-labeled oligonucleotides, which are complementary to bDNA amplifier sequences, bind to the bDNA molecule by hybridization. The bDNA signal is the chemiluminescent product of the AP reaction See, e.g., Tsongalis, Microbiol. Inf. Dis. 126:448-453, 2006; U.S. Pat. No. 7,033,758.

In further examples, the signal amplification method utilizes polymerized antibodies. In some examples, the labeled probe is detected by using a primary antibody to the label (such as an anti-DIG or anti-DNP antibody). The primary antibody is detected by a polymerized secondary antibody (such as a polymerized HRP-conjugated secondary antibody or an AP-conjugated secondary antibody). The enzymatic reaction of AP or HRP leads to the formation of strong signals that can be visualized.

It will be appreciated by those of skill in the art that by appropriately selecting labeled probe-specific binding agent pairs, multiplex detection schemes can be produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can be labeled with a first hapten, such as biotin, while a second probe that corresponds to a second target sequence can be labeled with a second hapten, such as DNP. Following exposure of the sample to the probes, the bound probes can be detected by contacting the sample with a first specific binding agent (in this case avidin labeled with a first fluorophore, for example, a first spectrally distinct QUANTUM DOT®, e.g., that emits at 585 nm) and a second specific binding agent (in this case an anti-DNP antibody, or antibody fragment, labeled with a second fluorophore (for example, a second spectrally distinct QUANTUM DOT®, e.g., that emits at 705 nm). Additional probes/binding agent pairs can be added to the multiplex detection scheme using other spectrally distinct fluorophores. Numerous variations of direct, and indirect (one step, two step or more) can be envisioned, all of which are suitable in the context of the disclosed probes and assays. Additional details regarding certain detection methods, e.g., as utilized in CISH and SISH procedures, can be found in Bourne, The Handbook of Immunoperoxidase Staining Methods, published by Dako Corporation, Santa Barbara, Calif.

B. Microarray Applications

Comparative genomic hybridization (CGH) is a molecular-cytogenetic method for the analysis of copy number changes (gain/loss) in the DNA content of cells. The contribution of genome structural variation to human disease is found in rare genomic disorders (for example, Trisomy 21, Prader-Willi Syndrome) and a broad range of human diseases, such as genetic diseases, autism, schizophrenia, cancers, and autoimmune diseases. In one example, the method is based on the hybridization of differently fluorescently labeled sample DNA (for example, labeled with fluorescein-FITC) and normal DNA (for example, labeled with rhodamine or Texas red) to normal human metaphase preparations. Using methods known in the art, such as epifluorescence microscopy and quantitative image analysis, regional differences in the fluorescence ratio of sample versus control DNA can be detected and used for identifying abnormal regions in the sample cell genome. CGH detects unbalanced chromosomes changes (such as increase or decrease in DNA copy number). See, e.g., Kallioniemi et al., Science 258:818-821, 1992; U.S. Pat. Nos. 5,665,549 and 5,721,098.

Genomic DNA copy number may also be determined by array CGH (aCGH). See, e.g., Pinkel and Albertson, Nat. Genet. 37:S11-S17, 2005; Pinkel et al., Nat. Genet. 20:207-211, 1998; Pollack et al., Nat. Genet. 23:41-46, 1999. Similar to standard CGH, sample and reference DNA are differentially labeled and mixed. However, for aCGH, the DNA mixture is hybridized to a slide containing hundreds or thousands of defined DNA probes (such as probes that specifically hybridize to a genomic target nucleic acid of interest). The fluorescence intensity ratio at each probe in the array is used to evaluate regions of DNA gain or loss in the sample, which can be mapped in finer detail than CGH, based on the particular probes which exhibit altered fluorescence intensity.

In general, CGH (and aCGH) does not provide information as to the exact number of copies of a particular genomic DNA or chromosomal region. Instead, CGH provides information on the relative copy number of one sample (such as a tumor sample) compared to another (such as a reference sample, for example a non-tumor cell or tissue sample). Thus, CGH is most useful to determine whether genomic DNA copy number of a target nucleic acid is increased or decreased as compared to a reference sample (such as a non-tumor cell or tissue sample) thereby determining the copy number variation of a target nucleic acid sample relative to a reference sample.

In a particular example, probes generated using the methods disclosed herein (for example, a probe including uniquely specific binding regions from one or more individual genes (including coding and/or non-coding portions of genes), one or more regions of a chromosome (e.g., regions include one or more genes of interest or no known genes) or even one or more entire chromosomes) may be utilized for aCGH. For example, an unlabeled probe prepared utilizing the methods described herein may be immobilized on a solid surface (such as nitrocellulose, nylon, glass, cellulose acetate, plastics (for example, polyethylene, polypropylene, or polystyrene), paper, ceramics, metals, and the like). Methods of immobilizing nucleic acids on a solid surface are well known in the art (see, e.g., Bischoff et al., Anal. Biochem. 164:336-344, 1987; Kremsky et al., Nuc. Acids Res. 15:2891-2910, 1987). As discussed above, differently fluorescently labeled sample DNA (for example, labeled with fluorescein-FITC) and reference DNA (for example, labeled with rhodamine or Texas red) is hybridized to the probe array and regional differences in the fluorescence ratio of sample versus reference DNA can be detected and used for identifying abnormal regions in the sample cell genome.

In another example, uniquely specific oligonucleotide probe nucleic acids designed as described herein are synthesized in situ on a solid surface (such as nitrocellulose, nylon, glass, cellulose acetate, plastics (for example, polyethylene, polypropylene, or polystyrene), paper, ceramics, metals, and the like). For example, uniquely specific segments defined using the methods described herein are utilized for printing, in situ, the oligonucleotide probes on a solid support utilizing computer based microarray printing methodologies, such as those described in U.S. Pat. Nos. 6,315,958; 6,444,175; and 7,083,975 and U.S. Pat. Application Nos. 2002/0041420, 2004/0126757, 2007/0037274, and 2007/0140906. In some examples, using a maskless array synthesis (MAS) instrument, oligonucleotides synthesized in situ on the microarray are under software control resulting in individually customized arrays based on the particular needs of an investigator. The number of uniquely specific oligonucleotides synthesized on a microarray varies, for example presently anywhere from 50,000 to 2.1 million probes, in various configurations, can be synthesized on a single microarray slide (for example, Roche NimbleGen CGH microarrays contain from 385,000 to 4 million or more probes/array).

Uniquely specific oligonucleotides probe sequences are synthesized either in situ by MAS instruments, or alternatively by utilizing photolithographic methods as described in U.S. Pat. Nos. 5,143,854; 5,424,186; 5,405,783; and 5,445,934.

Utilizing the disclosed uniquely specific probes for microarray applications is not limited by their method of manufacture, and a skilled artisan will understand additional methods of creating microarrays with uniquely specific oligonucleotide probes thereon that are equally applicable. For example, historical methods of spotting nucleic acid sequences onto solid supports are also contemplated, such that historically utilized nucleic acid probes are replaced by uniquely specific oligonucleotide probes as described herein. Regardless of method used to place probes on a microarray, the uniquely specific oligonucleotide probes can be used to target one or more nucleic acid samples, either individually or on the same array.

Applications of uniquely specific probes as designed herein that are in situ synthesized or otherwise immobilized on a microarray slide can be utilized for aCGH as well as other microarray based genomic target enrichment applications such as those described in U.S. Pat. Publication Nos. 2008/0194413, 2008/0194414, 2009/0203540, and 2009/0221438. Utilizing uniquely specific probes for generating in situ synthesized microarrays provides many improvements over current microarray probe designs. For example, use of uniquely specific probes allows for more specific binding of target sequences as compared to current probes, therefore not as many probes are needed per target and/or in conjunction more can be added to capture additional targets. Further, the need for blocking DNA (for example, Cot-1™ DNA) typically utilized in microarray experiments is reduced or eliminated when utilizing uniquely specific oligonucleotide probes.

For CGH applications, typically both target and reference genomic DNA are hybridized on one array for comparison on one microarray substrate. The CGH Analysis User's Guide (version 5.1, Roche NimbleGen, Madison, Wis.; available on the World Wide Web at nimblegen.com) describes methods for performing CGH analysis utilizing microarrays. In general, two genomic DNA samples, a target sample and a reference sample, are fragmented and labeled with different detection moieties (for example, Cy-3 and Cy-5 fluorescent moieties). The two labeled samples are mixed and hybridized to a microarray support, in this case a microarray comprising uniquely specific oligonucleotide probes, and the microarray is subsequently assayed for both detection moieties. The microarrays are scanned and detection data captured, for example by scanning a microarray with a microarray scanner (for example, a MS200 Microarray Scanner; Roche NimbleGen). The data is analyzed using analysis software (for example, NimbleScan; Roche NimbleGen). The target genomic sequence data is compared to the reference and DNA copy number gains and losses in target samples are thereby characterized. The target genomic sequences can be, for example, from targeted region(s) of one or more chromosome(s), one whole chromosome, or the total genomic complement of an organism (for example, a eukaryotic genome, such as a mammalian genome, for example a human genome).

For genomic enrichment (also known as sequence capture), typically a genomic sample is hybridized to a microarray support comprising targeted sequence specific probes for specific target enrichment prior to downstream applications, such as sequencing. The Sequence Capture User's Guide (version 3.1, Roche NimbleGen, incorporated by reference herein) describes methods for performing genomic enrichment. In general, a genomic DNA sample is prepared for hybridization to a microarray support, in this case a microarray comprising the disclosed uniquely specific oligonucleotide probes designed to capture targeted sequences from a genomic sample for enrichment. The captured genomic sequences are then eluted from the microarray support and sequenced, or used for other applications.

C. Blocking DNA

Genome-specific blocking DNA (such as human DNA, for example, total human placental DNA or Cot-1™ DNA) is usually included in a hybridization solution (such as for in situ hybridization or CGH) to suppress probe hybridization to repetitive DNA sequences or to counteract probe hybridization to highly homologous (frequently identical) off target sequences when a probe complementary to a human genomic target nucleic acid is utilized. In hybridization with standard probes, in the absence of genome-specific blocking DNA, an unacceptably high level of background staining (for example, non-specific binding, such as hybridization to non-target nucleic acid sequence) is usually present, even when a “repeat-free” probe is used. Nucleic acid probes produced by the methods disclosed herein exhibit reduced background staining, even in the absence of blocking DNA. In particular examples, the hybridization solution including the disclosed uniquely specific probe does not include genome-specific blocking DNA (for example, total human placental DNA or Cot-1™ DNA, if the probe is complementary to a human genomic target nucleic acid). This advantage is derived from the uniquely specific nature of the target sequences included in the nucleic acid probe; each labeled probe sequence binds only to the cognate uniquely specific genomic sequence. This results in dramatic increases in signal to noise ratios for ISH and CGH techniques.

Including blocking DNA in hybridization experiments not only adds an additional unwanted variable which can contribute to background staining, but it is also a costly component of hybridization experiments. In some examples, by utilizing uniquely specific probes generated using the methods of the present disclosure, experimental variability, background staining, and additional experimental cost can be bypassed.

In some examples the hybridization solution may contain carrier DNA from a different organism (for example, salmon sperm DNA or herring sperm DNA, if the genomic target nucleic acid is a human genomic target nucleic acid) to reduce non-specific binding of the probe to non-DNA materials (for example to reaction vessels or slides) with high net positive charge which can non-specifically bind to the negatively charged probe DNA.

### VIII. Methods of Producing and Using Uniquely Distinct Tags

Methods for producing nucleic acid tags specific to unique sequences of the genome are described herein. In some instances, it is desirable to create nucleic acid oligomers which are not present within a genome of interest, such as the human genome. Nucleic acid oligomers not specific to any portion of the genome of interest are not accurately described as probes since they do not hybridize to the genome. Instead, they are referred to herein as tags or as amplification sequences because they can be used to label other binding compounds, for example they can be bound to a uniquely specific probe. Because they are distinct from the genome, they are referred to herein as uniquely distinct tags. While the function or use of tags is vastly different than that of probes, the methods of producing tags are similar to those methods described herein for producing probes. As such, the disclosure herein related to probes is in many ways applicable to tags.

In illustrative embodiments, a method for producing nucleic acid tags, includes: (a) selecting a prospect nucleic acid sequence from a first genomic sequence, the first genomic sequence corresponding to genomic DNA for a divergent organism; (b) separating the prospect nucleic acid sequence into a plurality of segment sequences; (c) comparing the plurality of segment sequences to a second genomic sequence, the second genomic sequence corresponding to genomic DNA for an organism of interest; (d) selecting a plurality of segment sequences not homologous to any region of the second genomic sequence from the plurality of segment sequences; (e) preparing a plurality of test oligonucleotides corresponding to the plurality of segment sequences not homologous to any region of the second genomic sequence; (f) testing hybridization of the plurality of test oligonucleotides against the genomic DNA for the organism of interest; (g) selecting a plurality of tag sequences identified in the hybridization testing as uniquely distinct from the genomic DNA for the organism of interest; and (h) preparing the nucleic acid tags using one or more of the plurality of nucleic acid tag sequences identified in the hybridization testing as uniquely distinct from the genomic DNA for the organism of interest.

In some aspects, selecting a prospect nucleic acid sequence from a first genomic sequence is analogous to selecting a target nucleic acid sequence. However, the prospect nucleic acid sequence differs from the target nucleic acid in that it is selected purposefully to include sequences that are not found within the genome of the organism of interest. One manner of selecting a prospect genomic sequence is to utilize the myriad of sequences nature has produced in the vast genomic diversity of the various species. Accordingly, a candidate genomic sequence could be selected from a genome different than the genome of interest. For example, a source of tags uniquely distinct from the human genome would be a non-human genome, such as rice (Oryza) or other plant (e.g., Arabidopsis) or fruit fly (Drosophila) genome. The genotypic diversity between species insures a vast number of uniquely distinct sequences. In illustrative embodiments, the organism of interest is a mammal, such as a human. In one embodiment, the divergent organism is one having less than about 95% sequence homology with the organism of interest, such as less than 90%, less than 80%, less than 75%, less than 70%, less than 60%, less than 50%, or less than 40% sequence homology with the organism of interest, for example 10% to 90%, 10 to 80%, or 10 to 50% sequence homology with the organism of interest. In specific embodiments, the divergent organism is Oryza, Arabidopsis, C. elegans, or Drosophila.

In some examples, separating the prospect nucleic acid sequence into a plurality of segment sequences is analogous to the separation step described herein for producing nucleic acid probes. However, since selecting a target nucleic acid sequence can be from much larger genomic sources (e.g. the entire genome of a divergent species compared to an exemplary 500,000 bp target region such as one from a human genome), the step of separating the prospect nucleic acid sequence into segment may be modified to account for the potentially much larger sequence set. In one embodiment, the plurality of segment sequences are at least 20 nucleotides in length, such as at least 50, at least 100, at least 200, or at least 500 nucleotides in length, such as about 20-500 or 20 to 100 nucleotides in length. In another embodiment, the plurality of segment sequences overlap by at least about 10 nucleotides, such as at least 20, at least 50, at least 100, or at least 200 nucleotides. In another embodiment, comparing the plurality of segment sequences to the second genomic sequence and/or selecting the plurality of segment sequences not homologous to any region of the second genome from the plurality of segment sequences is done in silico.

Comparing the plurality of segment sequences to a genomic sequence can be done as described herein. One aspect of the uniquely distinct tags is that the genomic sequence segments from the divergent genome are compared to genomic DNA for an organism of interest. The prospect segments are compared to the genome of the organism of interest with an objective of identifying those sequences that are not present in the organism of interest. From this identification, a set of sequences can be selected that appear uniquely distinct using bioinformatics approach. The uniquely distinct aspect is verified by synthesizing test oligonucleotides so that empirical testing can confirm the validity of the uniquely distinct aspect for each sequence. As described herein, synthesizing test oligonucleotides can be done using methods known in the art, such as a solid-phase technique, for example a microarray. Synthesizing the test oligonucleotides on a microarray enables facilitates hybridization testing which can include contacting the microarray with genomic DNA for the organism of interest. Using this approach, test oligonucleotides that exhibit no hybridization against the genomic DNA for the organism of interest are empirically established as uniquely distinct from the genome. In some examples, the plurality of nucleic acid tag sequences identified in the hybridization testing as uniquely distinct are at least 50 nucleotides in length, such as at least 75, at least 100, at least 100, or at lest 500 nucleotides in length.

In illustrative embodiments, the method of producing uniquely distinct nucleic acid tags includes preparing the nucleic acid tags. Preparing the tags can be done by any technique known or developed. One such method includes synthesizing the nucleic acid tags using oligonucleotide synthesis, such as a solid-phase synthesis. In another embodiment, preparing the nucleic acid tags includes joining the plurality of nucleic acid tag sequences identified in the hybridization testing as uniquely distinct. In another embodiment, preparing the nucleic acid tags includes joining the plurality of nucleic acid tag sequences identified in the hybridization testing as uniquely distinct using a joining method such as by enzymatically joining by using a ligase in a ligation reaction, enzymatically joining by using a recombinase in a recombination reaction, chemically joining by using modified nucleotides, or joining by using an amplification reaction. In yet another embodiment, preparing the nucleic acid tags includes introducing the plurality of nucleic acid tag sequences identified in the hybridization testing as uniquely distinct into a vector and replicating. In one embodiment, separating the genomic target nucleic acid sequence into a plurality of segment sequences includes eliminating those segment sequences not having G/C nucleotide content between about 30% and 70%. In another embodiment, testing hybridization of the plurality of test oligonucleotides to the genomic DNA includes using an array of the plurality of test oligonucleotides. In yet another embodiment, testing hybridization of the plurality of test oligonucleotides to the genomic DNA includes establishing a mathematical model for hybridization scores of total genomic DNA and blocking DNA and establishing one or more predetermined cutoffs.

The resulting nucleic acid tag generated using these methods can be labeled or used as a label on a binding moiety (either with a detectable moiety, such as a fluorophore, or with a moiety such as a hapten). The tag may also be labeled with a second tag or binding moiety. For example, the oligonucleotide can be synthesized using oligonucleotide synthesis in combination with a uniquely specific probe. For example, one oligonucleotide may include a uniquely specific probe portion and a uniquely distinct tag section. Because a uniquely distinct tag was selected, the overall nucleic acid oligomer will only bind to that unique location on the genome of interest without interfering hybridization from the uniquely distinct region of the oligonucleotide. After labeling a target with a uniquely distinct tag, there are a myriad of techniques available to detect or quantify the presence of the tag. In one example, an oligonucleotide having at least a portion of the sequence complimentary to the tag can be introduced into the solution. This oligonucleotide can then hybridize to the tag without hybridizing to any other sequences within the sample. This complimentary sequence can include features which facilitate detection and quantification, such as fluorophores, lumiphores, haptens, or the like. Detection of the secondary labels can be either done directly or indirectly.

### IX. Kits

Kits including at least one nucleic acid probe including at least two binding regions complementary to uniquely specific nucleic acid sequences generated as described herein are also a feature of this disclosure. In addition, kits including at least one nucleic acid tag generated as described herein are also a feature of this disclosure. For example, kits including at least one nucleic acid segment or tag uniquely distinct from the genomic DNA for the organism of interest are also disclosed. For example, kits for in situ hybridization or array CGH include at least one nucleic acid segment or tag not represented in a target genome as described herein. In some examples, kits include one or more nucleic acid molecules not represented in a target genome generated using the methods disclosed herein.

For example, kits for in situ hybridization procedures such as FISH, CISH, and/or SISH include at least one probe or tag (such as at least two, at least three, at least five, or at least 10 probes and/or tags) as described herein. In another example, kits for array CGH include at least one probe or tag as described herein. Accordingly, kits can include one or more nucleic acid probes including at least two binding regions complementary to uniquely specific nucleic acid sequences generated using the methods disclosed herein.

The kits can also include one or more reagents for performing an in situ hybridization or CGH assay, or for producing a probe or tag. For example, a kit can include at least one uniquely specific nucleic acid probe or tag (or population of such probes or tags), along with one or more buffers, labeled dNTPs, a labeling enzyme (such as a polymerase), primers, nuclease free water, and instructions for producing a labeled probe or tag.

In one example, the kit includes one or more uniquely specific nucleic acid probes (unlabeled or labeled) along with buffers and other reagents for performing in situ hybridization. For example, if one or more unlabeled uniquely specific nucleic acid probes are included in the kit, labeling reagents can also be included, along with specific detection agents and other reagents for performing an in situ hybridization assay, such as paraffin pretreatment buffer, protease(s) and protease buffer, prehybridization buffer, hybridization buffer, wash buffer, counterstain(s), mounting medium, or combinations thereof. In some examples, such kit components are present in separate containers.

The kit can optionally further include control slides for assessing hybridization and signal of the probe.

In certain examples, the kits include avidin, antibodies, and/or receptors (or other anti-ligands). Optionally, one or more of the detection agents (including a primary detection agent, and optionally, secondary, tertiary or additional detection reagents) are labeled, for example, with a hapten or fluorophore (such as a fluorescent dye or QUANTUM DOT®). In some instances, the detection reagents are labeled with different detectable moieties (for example, different fluorescent dyes, spectrally distinguishable QUANTUM DOT®s, different haptens, etc.). For example, a kit can include two or more different uniquely specific nucleic acid probes that correspond to and are capable of hybridizing to different genomic target nucleic acid sequences (for example, any of the target sequences disclosed herein). The first probe can be labeled with a first detectable label (e.g., hapten, fluorophore, etc.), the second probe can be labeled with a second detectable label, and any additional probes (e.g., third, fourth, fifth, etc.) can be labeled with additional detectable labels. The first, second, and any subsequent probes can be labeled with different detectable labels, although other detection schemes are possible. If the probe(s) are labeled with indirectly detectable labels, such as haptens, the kits can include detection agents (such as labeled avidin, antibodies or other specific binding agents) for some or all of the probes. In one embodiment, the kit includes probes and detection reagents suitable for multiplex ISH.

In one example, the kit also includes an antibody conjugate, such as an antibody conjugated to a label (e.g., an enzyme, fluorophore, or fluorescent nanoparticle). In some examples, the antibody is conjugated to the label through a linker, such as PEG, 6×-His, streptavidin, and GST.

In another example, the kit includes one or more uniquely specific nucleic acid probes affixed to a solid support (such as an array) along with buffers and other reagents for performing CGH. Reagents for labeling sample and control DNA can also be included, along with other reagents for performing an aCGH assay, prehybridization buffer, hybridization buffer, wash buffer, or combinations thereof. The kit can optionally further include control slides for assessing hybridization and signal of the labeled DNAs.

The disclosure is further illustrated by the following non-limiting Examples.

### EXAMPLES

### Example 1

### Generation of Uniquely Specific Gene Probes

This example describes the design and production of a gene probe consisting of uniquely specific nucleic acid sequences.

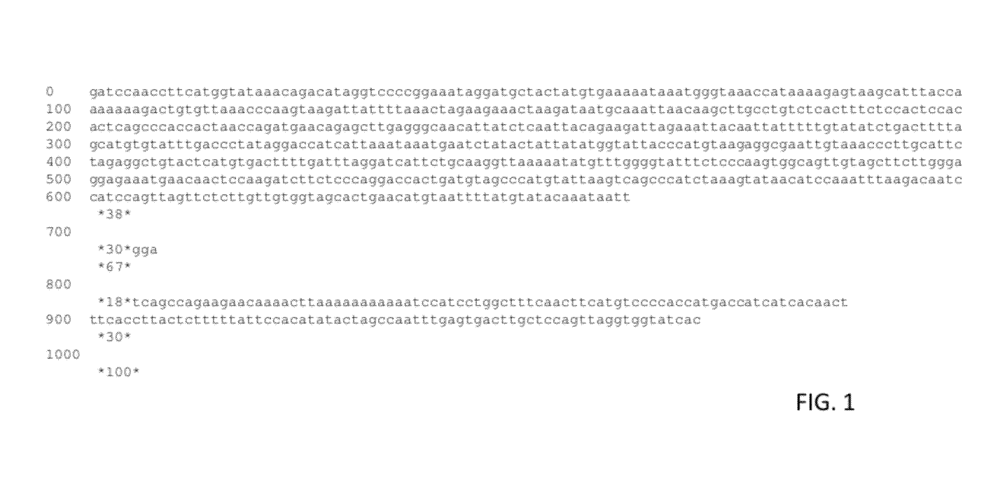

To generate a uniquely specific gene probe, an approximately 700,000 bp region of human chromosome 7q31.2 including the MET gene located between base pairs 115809695-116513594 (using the March 2006 [hg18] build of the human genome; UCSC Genome browser; genome.ucsc.edu) was selected. The sequence was screened to identify repetitive nucleic acid sequences using RepeatMasker, enumerated, and separated into 100 bp segments with the repetitive sequences replaced by the number of by within the repetitive element (FIG. 1). The repeat-free 100 bp segments within the region were then analyzed with BLAT (BLAST-Like Alignment Tool). Segments that did not have any sequence identity to any other region of chromosome 7 or any other human chromosome were identified as uniquely specific nucleic acid sequences.

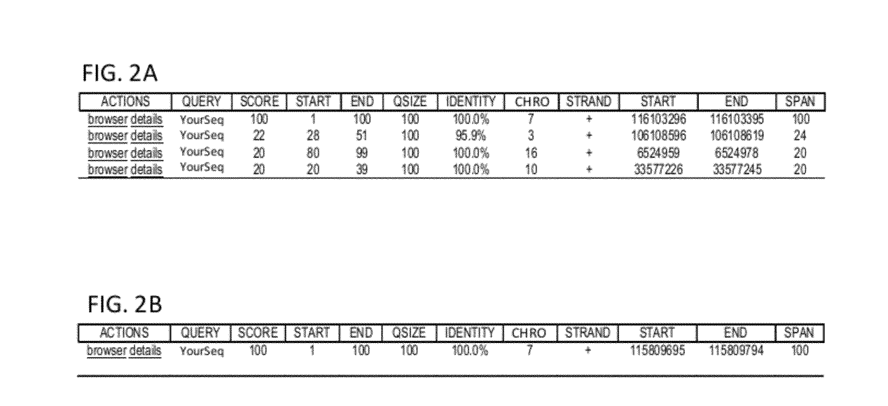

For example, a 100 bp segment (nucleotides 116103296-116103395 of chromosome 7) had regions of sequence identity to sequences on chromosomes 3, 16, and 10 (FIG. 2A). Therefore, this sequence is not a uniquely specific nucleic acid sequence and was not included in the uniquely specific gene probe. In contrast, another 100 bp segment (nucleotides 115809695-115809794 of chromosome 7) did not have any regions of sequence identity to any other region of the human genome (FIG. 2B). Therefore, this sequence is a uniquely specific nucleic acid sequence, which was included in the uniquely specific gene probe.

TABLE 1
Summary of uniquely specific MET probe sequences

| | Size of Plasmid | | | | |
|---|---|---|---|---|---|
| | Insert (Probe | Identity | Chr 7 bp | Chr 7 bp | Chromosomal |
| Plasmid Name | Length) | with Chr 7 | Start | End | Span (bp span) |
| MET Plasmid 1 | 5500 | 100.00% | 115809695 | 116504794 | 695,099 |
| MET Plasmid 2 | 5499 | 100.00% | 115812695 | 116505594 | 692,899 |
| MET Plasmid 3 | 5500 | 100.00% | 115817594 | 116512994 | 695,400 |
| MET Plasmid 4 | 5300 | 100.00% | 115820694 | 116513194 | 692,500 |
| MET Plasmid 5 | 5400 | 100.00% | 115822495 | 116513594 | 691,099 |
| TOTAL | 27199 | 100.00% | | | 703,899 |

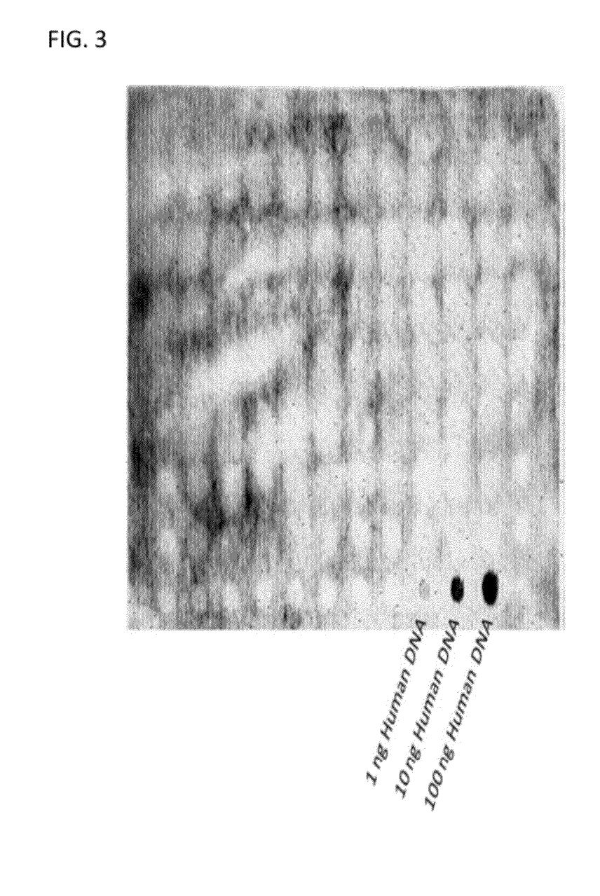

Following one pass of the 700,000 base pair region, 273 uniquely specific 100 bp sequences were identified. Each of the uniquely specific 100 bp sequences was synthesized as an oligonucleotide. Each oligonucleotide was spotted on a membrane (15 μg oligonucleotide per spot). The membrane was prehybridized for 2 hours at 42° C. with a buffer containing 50% formamide and 1 mg/ml salmon sperm DNA (Life Technologies, Carlsbad, Calif.). A nick-translated human placental DNA probe (labeled with DNP-dCTP through nick-translation; Sambrook et al., Molecular Cloning: A Laboratory Manual, 2^{nd} ed., Cold Spring Harbor Laboratory Press, 1989, substituting hapten-labeled dCTP for ³²P-dNTP) was added at a final concentration of 1 μg/ml, and incubated for 18 to 24 hours at 42° C. Following probe hybridization, the membranes were washed three times in a buffer containing 2×SSC with 1% Brij 35 at 42° C. The probe hybridization was detected using the CDP Star detection kit from Sigma-Aldrich (St. Louis, Mo.), using an alkaline phosphatase conjugated mouse monoclonal anti-DNP antibody (Sigma-Aldrich, Cat. No. 066K4842). The probe did not hybridize with any of the oligonucleotides (FIG. 3), indicating that all the identified sequences were uniquely specific to the human genome.

The sequences were initially organized in five approximately 5500 bp segments. The sequences were organized in the order that they occurred in the target and then placed in the plasmids such that the first plasmid contained sequences 1, 6, 11, 16, and so on; the second plasmid contained sequences 2, 7, 12, 17 and so on; the third plasmid contained sequences 3, 8, 13, 18, and so on; the fourth plasmid contained sequences 4, 9, 14, 19, and so on; and the fifth plasmid contained sequences 5, 10, 15, 20, and so on. Each of the initially ordered 5500 bp segments was analyzed using BLAT to determine if any non-uniquely specific nucleic acid sequences were produced. One of the initial 5500 bp segments resulted in a non-uniquely specific nucleic acid sequence. The 100 bp segment that produced the non-uniquely specific nucleic acid sequence was moved to the 3′ end of the order; this placement resulted in a 5500 bp segment that consisted only of uniquely specific nucleic acid sequence.

Each 5500 bp sequence was synthesized in vitro (GeneArt, Regensburg, Germany) and inserted into a modified pUC plasmid backbone. Five plasmids containing a total of 27,199 bp of sequence were generated. The plasmids were pooled together in an equimolar ratio and labeled by nick translation for use for in situ hybridization (see Example 2). The nick translation reaction included 8 U DNA polymerase I (Roche Applied Science) and 0.0025 U DNaseI (Roche Applied Science) per microgram of DNA, 3 mM MgCl₂, and 2:1 DNP-dCTP:dCTP (66 μM:34 μM) and was incubated at 22° C. for 17 hours. An approximately 1,000,000 bp region of human chromosome 15q26 was selected to generate an IGF1R probe. Sequence analysis, dot-blotting, and ordering were performed as described for the MET probe. The plasmids generated are as shown in Table 2.

TABLE 2
Summary of uniquely specific IGF1R probe sequences

| | Size of Plasmid | Identity | Chr. 15 | Chr. 15 | Chromosomal |
|---|---|---|---|---|---|
| | Insert (Probe | with Chr. | base pair | base pair | Span (base pair |
| Plasmid Name | Length) | 15 | Start | End | span) |
| IGF1R Plasmid1 | 5300 | 100.00% | 96661884 | 96826583 | 164,700 |
| IGF1R Plasmid2 | 5303 | 100.00% | 96828084 | 97015583 | 187,500 |
| IGF1R Plasmid3 | 5300 | 100.00% | 97016784 | 97107783 | 91,000 |
| IGF1R Plasmid4 | 5300 | 100.00% | 97112884 | 97216783 | 103,900 |
| IGF1R Plasmid5 | 5200 | 100.00% | 97216984 | 97309083 | 92,100 |
| IGF1R Plasmid6 | 5000 | 100.00% | 97309584 | 97481983 | 172,400 |
| IGF1R Plasmid7 | 5200 | 100.00% | 97482284 | 97674883 | 192,600 |
| TOTAL | 36,603 | 100.00% | | | 1,012,999 |

An approximately 1,000,000 bp region of human chromosome 12p12.1 was selected to generate a KRAS probe. Sequence analysis, dot-blotting, and ordering were performed as described for the MET probe. The plasmids generated are as shown in Table 3.

TABLE 3
Summary of uniquely specific KRAS probe sequences

| | Size of Plasmid | Identity | Chr. 12 | Chr. 12 | Chromosomal |
|---|---|---|---|---|---|
| | Insert (Probe | with Chr. | base pair | base pair | Span (base pair |
| Plasmid Name | Length) | 12 | Start | End | span) |
| KRAS Plasmid1 | 5300 | 100.00% | 25610831 | 25783130 | 172,300 |
| KRAS Plasmid2 | 5600 | 100.00% | 25426731 | 25601430 | 174,700 |
| KRAS Plasmid3 | 5500 | 100.00% | 25265931 | 25425430 | 159,500 |
| KRAS Plasmid4 | 5500 | 100.00% | 25045731 | 25261430 | 215,700 |
| KRAS Plasmid5 | 5500 | 100.00% | 24886231 | 25042430 | 156,200 |
| KRAS Plasmid6 | 5500 | 100.00% | 24788631 | 24885730 | 971,00 |
| TOTAL | 33,100 | 100.00% | | | 994,499 |

An approximately 1,000,000 bp region of human chromosome 18p11.32 was selected to generate a TS probe. Sequence analysis, dot-blotting, and ordering were performed as described for the MET probe. The plasmids generated are as shown in Table 4.

TABLE 4
Summary of uniquely specific TS probe sequences

| | Size of Plasmid | Identity | Chr. 18 | Chr. 18 | Chromosomal |
|---|---|---|---|---|---|
| | Insert (Probe | with Chr. | base pair | base pair | Span (base pair |
| Plasmid Name | Length) | 18 | Start | End | span) |
| TS Plasmid 1 | 4858 | 100.00% | 649404 | 763303 | 113,900 |
| TS Plasmid 2 | 4859 | 100.00% | 763304 | 895303 | 132,000 |
| TS Plasmid 3 | 4859 | 100.00% | 896704 | 1040903 | 144,200 |
| TS Plasmid 4 | 4855 | 100.00% | 1063804 | 1294103 | 230,300 |
| TS Plasmid 5 | 4855 | 100.00% | 1294804 | 1480703 | 185,900 |
| TS Plasmid 6 | 4460 | 100.00% | 1490104 | 1642803 | 152,700 |
| TOTAL | 28,746 | 100.00% | | | 993,399 |

### Example 2

### Comparison of Uniquely Specific Probes with Repeat-Free Probes

This example compares the performance of uniquely specific probes and repeat-free probes for in situ hybridization.

The uniquely specific MET probe was prepared as described in Example 1. The repeat-free MET probe was prepared by PCR amplifying 156 non-repetitive DNA sequences within a 500,000 bp region of chromosome 7q31.2. The repeat free MET probe has an overall coverage of approximately 425,000 bp on chromosome 7 at 7q31.2, which includes the MET gene sequence. Following the PCR, the purified amplicons were screened using a dot blot, as described in Example 1. The PCR fragments that did not hybridize to the human DNA probe were pooled together at an equal molar concentration, and randomly ligated together using DNA ligase. The resulting ligated concatenated DNA product was amplified using Whole Genome Amplification (Qiagen, Valencia, Calif.).

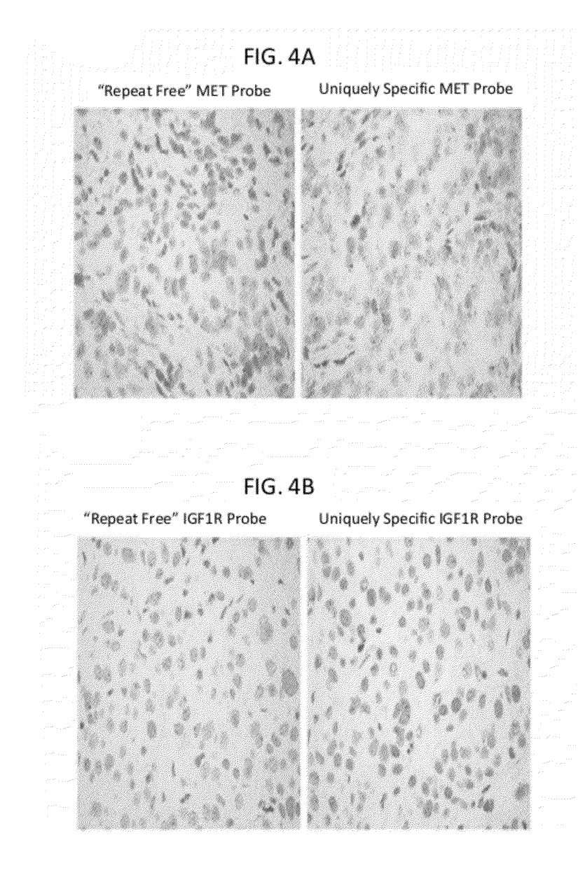

Both the uniquely specific probe and a repeat-free probe were used on the Ventana BENCHMARK XT with silver in situ hybridization (SISH) detection. The probes were labeled with DNP-dCTP using nick-translation as described in Example 1. The repeat-free probe was used at a concentration of 10 μg/ml with 2 mg/ml human placental blocking DNA (FIG. 4A, left panel). The uniquely specific probe was used at a concentration of 20 μg/ml with 1 mg/ml sheared salmon sperm DNA (Life Technologies) (FIG. 4A, right panel). Staining with the uniquely specific probe was comparable to staining with the repeat-free probe, however human DNA blocking reagent was not required.

The uniquely specific IGF1R probe was prepared as described in Example 1. The repeat-free IGF1R probe was prepared by PCR amplifying 200 non-repetitive DNA sequences within a 500,000 bp region of chromosome 15q26.3. Following the PCR, the purified amplicons were screened using a dot blot, as described in Example 1. The PCR fragments that did not hybridize to the human DNA probe were pooled together at an equal molar concentration, and randomly ligated together using DNA ligase. The resulting ligated, concatenated DNA product was amplified using Whole Genome Amplification (Qiagen).

Both the uniquely specific IGF1R probe and the repeat-free IGF1R probe were used on the Ventana BENCHMARK XT with silver in situ hybridization (SISH) detection. The probes were labeled with DNP-dCTP using nick-translation as described in Example 1. The repeat-free IGF1R probe was used at a concentration of 10 μg/ml with 2 mg/ml whole male placental human DNA (FIG. 4B, left panel). The uniquely specific IGF1R probe was used at a concentration of 30 μg/ml with 0.25 mg/ml human placental blocking DNA and 1.75 mg/ml sheared salmon sperm DNA (FIG. 4B, right panel).

### Example 3

### Comparison of Probe Hybridization with and without Blocking DNA

This example describes experiments demonstrating that blocking DNA is not required when using the uniquely specific probes of the present disclosure in in situ hybridizations.

Lung cancer test tissue array slides were obtained from US Biomax, Inc. (Rockville, Md.; Cat. No. TMA-T044). Uniquely specific probes to MET, IGF1R, KRAS, and TS were generated as described in Example 1.

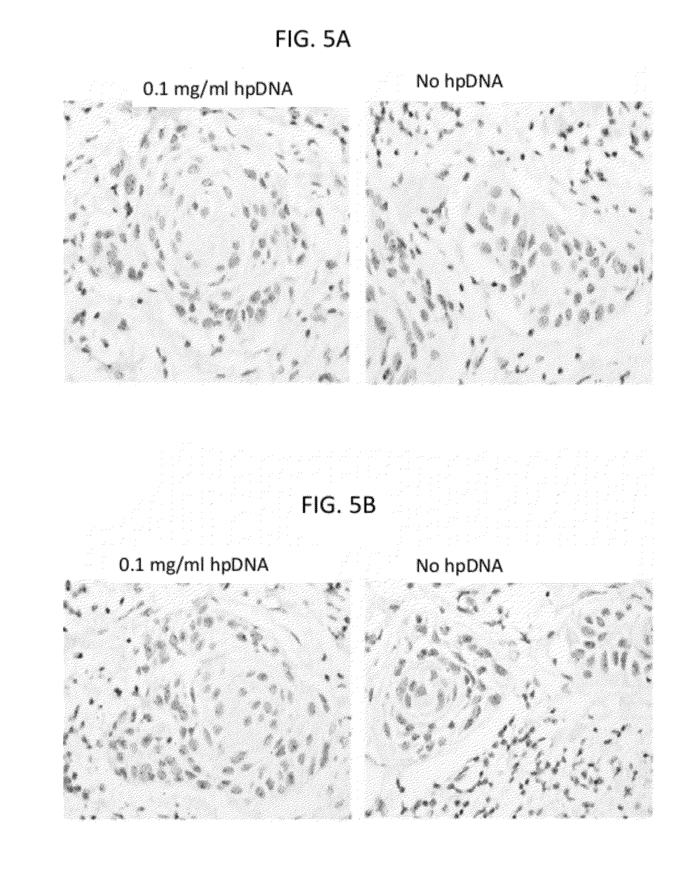

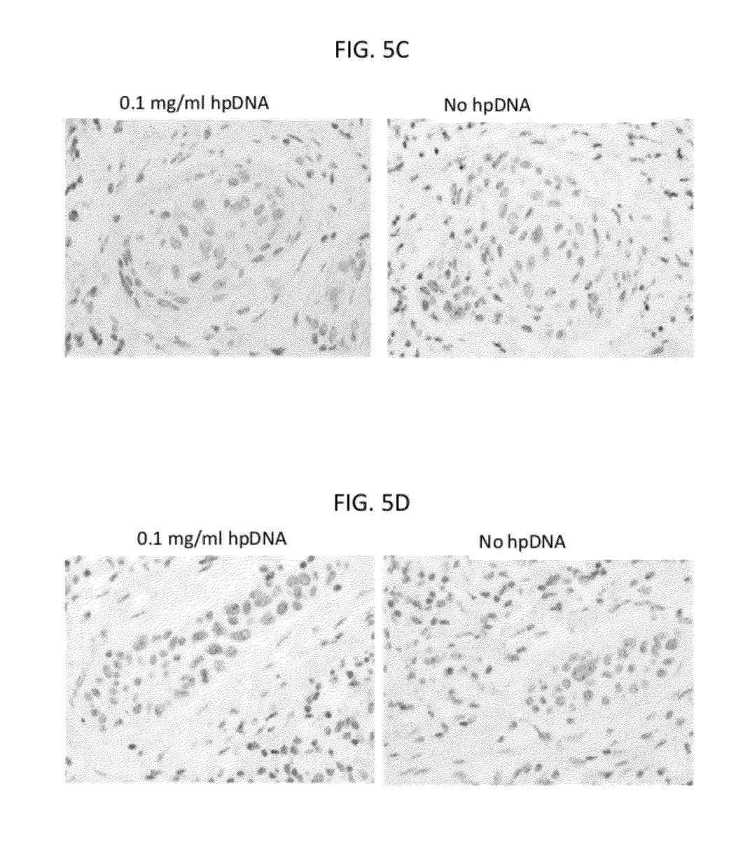

Lung cancer slides were processed and stained on the BENCHMARK XT system (Ventana Medical Systems) and detected by SISH detection. In situ hybridizations were performed with 10 μg/ml of nick-labeled uniquely specific probe DNA with or without 0.1 mg/ml human placental blocking DNA (hpDNA) in the presence of carrier DNA (herring DNA at 1 mg/ml; Roche Diagnostics). As seen in FIGS. 5A-D, when using the uniquely specific probes, there was no need for blocking DNA during hybridization. In general, probe signal was equivalent, or even better, when human blocking DNA was omitted.

### Example 4

### Generation of Uniquely Specific Probes Utilizing Empiric Selection

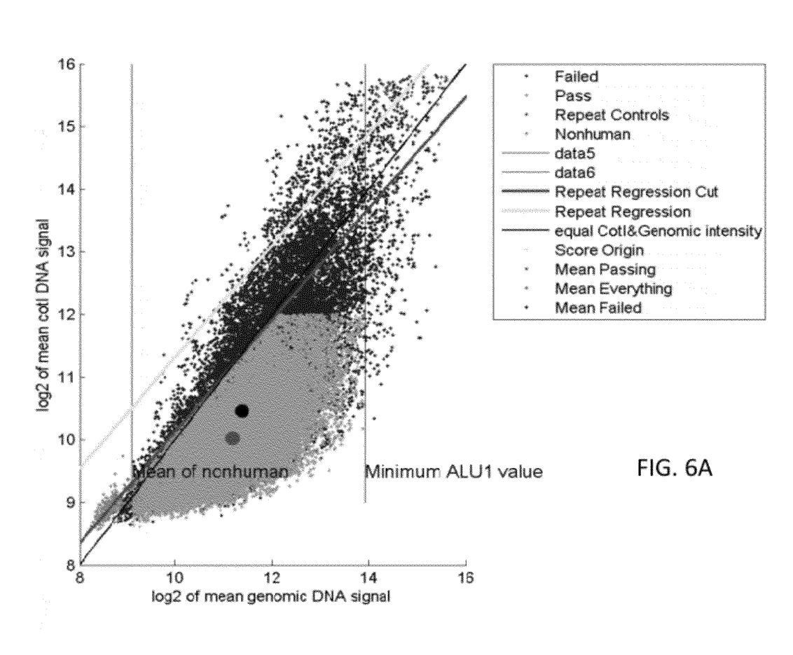

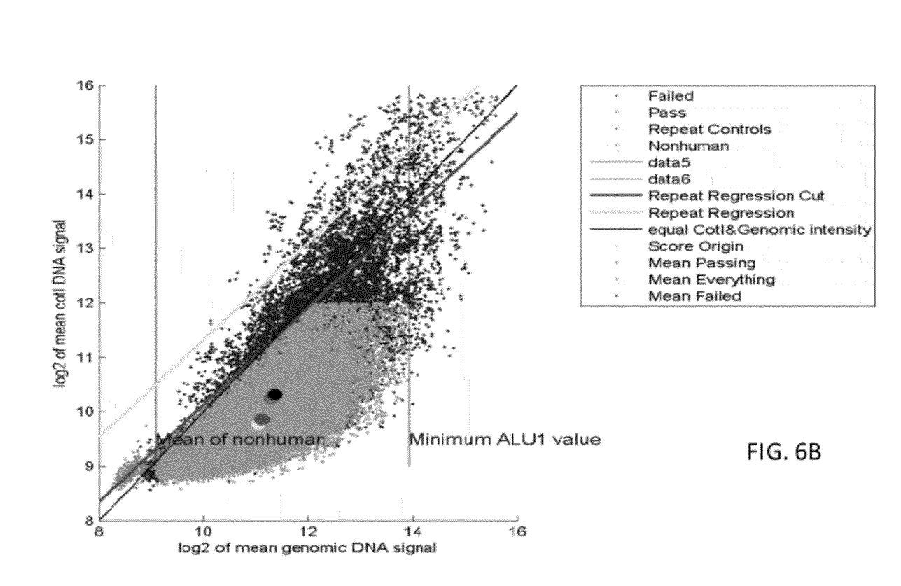

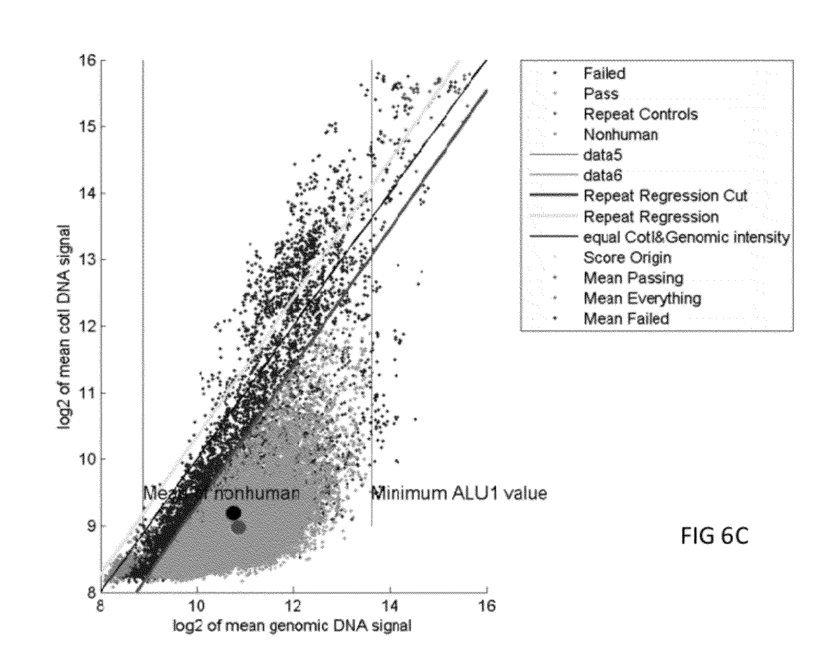

An approximately 1,000,000 bp region of human chromosome 11q31.2 was selected to generate a CCND1 probe. MATLAB® software was used to separate the acquired target sequence into 100 bp sequences, tiling by 10 bp. Following the enumeration of an 100 bp candidate sequences, the percentage of guanosine and cytosine was determined in MATLAB® and an sequences above 65% and below 35% were eliminated. The remaining candidate 100 bp sequences were printed on a NimbleGen 2.1M CGH slide and probed simultaneously with a total human genomic probe, and a Cot-1™ DNA probe according to NimbleGen processes. Positive controls (positive DNA sequences were ALU1, D17Z1 alpha satellite, the Sau3 LINE element, and the pHuR93Telo telomeric repetitive element) and negative controls (DNA sequences from the rice genome) were included on the array to establish cutoffs for selection criteria. Fifty-eight rice genome sequences were selected from chromosome 5 (base pairs 20,000,000 to 21,000,000) of Oryza sativa. Data acquisition and normalization were provided by NimbleGen. MATLAB® was used to analyze the NimbleGen data and establish sequence selection criteria by deriving a linear regression of an the positive control sequences, followed by decreasing the linear regression by one standard deviation. The cut off for the negative controls (rice DNA sequences) was established by using the mean of the total human genomic DNA score of the negative control sequences. Two additional cut offs were created by using the minimum human genomic score from the ALU1 sequences, and a hard cut of for the Cot-™ score was set at 12 (FIG. 6A).

MATLAB® was then utilized to eliminate overlapping candidate sequences. Five hundred 100 bp uniquely specific candidate sequences were organized into 5000 bp concatenated sequences in the order they appear on the genomic target. The 5000 bp sequences were then synthesized in vitro (GeneWiz, South Plainfield, N.J.) and inserted into a modified pUC plasmid backbone. Ten plasmids each containing 5000 bp of sequences were synthesized.

An approximately 1,000,000 bp region of human chromosome 12q14.1 was selected to generate a CDK4 probe. Sequence analysis, array analysis, and ordering were performed as described for the CCND1 probe (FIG. 6B).

An approximately 1,000,000 bp region of human chromosome 6q23.3 was selected to generate a Myb probe. Sequence analysis, array analysis, and ordering were performed as described for the CCND1 probe (FIG. 6C).

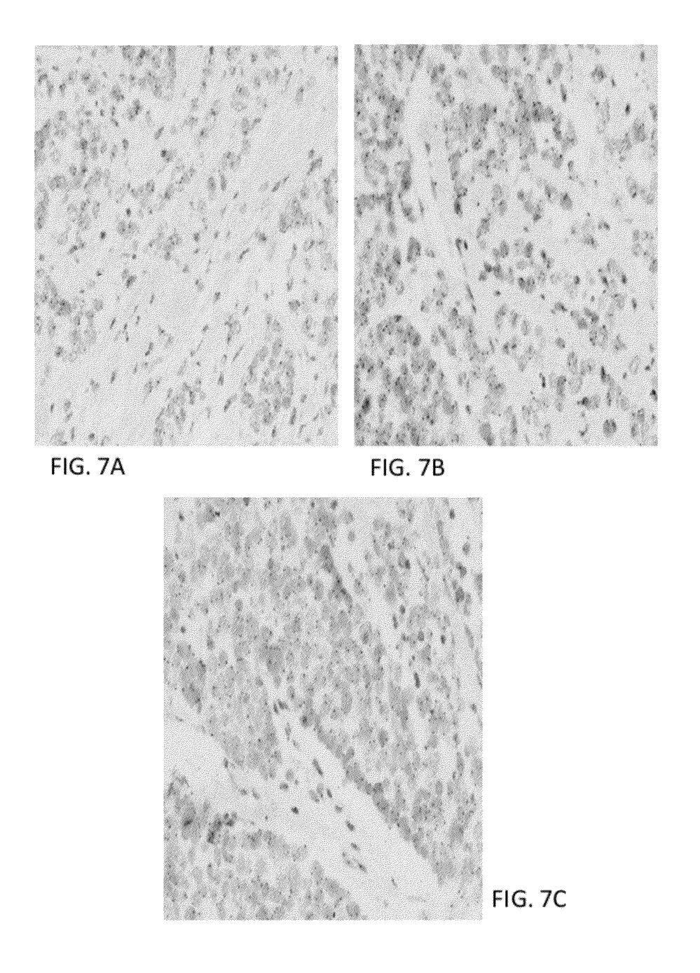

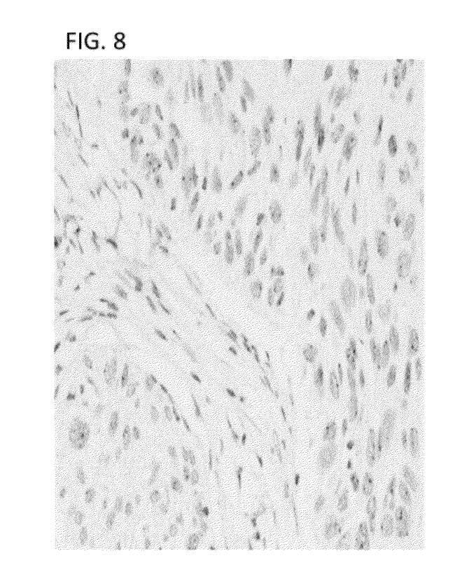

Plasmid pooling, labeling and staining with each of the probes was performed as described for the MET probe (Example 1). Each probe was hybridized to a BioMax lung cancer array without use of human placental blocking DNA, and detected using SISH (FIG. 7A-C).

### Example 5

### In situ Hybridization with a Single Plasmid Probe

An approximately 60,000 bp region of human chromosome 7p11.2 was selected to generate an EGFR probe. Sequence analysis, array analysis, and ordering were performed as described for the CCND1 probe (Example 4), with the exception that only a single 5000 bp plasmid was used as the probe. The EGFR probe (5 μg/ml) was hybridized to a BioMax lung cancer array without use of human placental blocking DNA, and detected using HRP activated tyramide conjugated to hydroxyquinoxaline (HQ), followed by SISH detection with an anti-HQ monoclonal antibody conjugated to HRP (FIG. 8).

### Example 6

### Microarray Methods

This example describes methods for comparing performance of uniquely specific probes generated using the methods described herein with repeat-free probes generated by previously utilized methods hybridized to a comparative genomic hybridization (CGH) array.

A uniquely specific probe is generated as described in Example 1 or Example 4 (for example, an epidermal growth factor receptor (EGFR) probe). A repeat-free probe that hybridizes to the same target nucleic acid (such as EGFR) is generated by methods previously known in the art (for example, the methods described in Example 2). Individual binding regions (uniquely specific segments) from the uniquely specific probe are printed on one CGH array. Individual repeat-free segments from the repeat-free probe are printed on a second CGH array.

CGH is performed using routine methods (e.g., NimbleGen Array User's Guide, CGH Analysis version 4.0, Roche NimbleGen, Madison, Wis.). Genomic DNA samples are prepared and labeled (for example, with Cy₃ or Cy₅). The labeled genomic DNA is hybridized to each of the CGH arrays. Appropriate stringency washes are performed following hybridization. The array is then scanned (for example, using a GenePix 4000B scanner) and the data is analyzed (for example, with NimbleScan software).

Hybridization with the uniquely specific probe array is comparable to hybridization with the repeat-free probe array.

### Example 7

### Diagnostic Methods

This example describes particular methods that can be used for determining a diagnosis or prognosis of a subject (such as a subject with cancer) utilizing probes generated by the methods described herein. However, one skilled in the art will appreciate that methods that deviate from these specific methods can also be used to successfully provide a diagnosis or prognosis of a subject.

A sample, such as a tumor sample, is obtained from the subject. Tissue samples are prepared for ISH, including deparaffinization and protease digestion.

In one example, the diagnosis of a tumor (for example, a lung tumor, such as a non-small cell lung carcinoma (NSCLC)) is determined by determining MET gene copy number by in situ hybridization in a tumor sample obtained from a subject. For example, the sample, such as a tissue or cell sample present on a substrate (such as a microscope slide) is incubated with a MET probe complementary to uniquely specific nucleic acid sequence, such as a MET probe generated as described in Example 1. The hybridization is carried out in the absence of human DNA blocking reagent (for example, in the absence of Cot-1™ DNA). Hybridization of the MET probe to the sample is detected, for example, using microscopy. The MET gene copy number is determined by counting the number of MET signals per nucleus in the sample and calculating an average MET gene copy number/cell. An increase in MET gene copy number/cell in the tumor sample (such as a MET gene copy number of more than 2, 3, 4, 5, 10, 20, or more) or an increase in MET gene copy number relative to a control (such as a non-neoplastic sample or a reference value) indicates a diagnosis of cancer (such as NSCLC). In contrast, no substantial change in MET gene copy number (such as an MET gene copy number of about 2 or less) or no substantial change in MET gene copy number relative to a control (such as a non-neoplastic sample or a reference value) does not indicate a diagnosis of cancer (such as the absence of NSCLC).

In another example, the prognosis of a tumor (for example, a lung tumor, such as a NSCLC) is determined by determining IGF1R gene copy number by in situ hybridization in a tumor sample obtained from a subject. For example, the sample, such as a tissue or cell sample present on a substrate (such as a microscope slide) is incubated with a IGF1R probe complementary to uniquely specific nucleic acid sequence, such as an IGF1R probe generated as described in Example 1. The hybridization is carried out in the absence of human DNA blocking reagent (for example, in the absence of Cot-1™ DNA). Hybridization of the IGF1R probe to the sample is detected, for example, using microscopy. The IGF1R gene copy number is determined by counting the number of IGF1R signals per nucleus in the sample and calculating an average IGF1R copy number/cell. An increase in IGF1R gene copy number/cell in the tumor sample (such as an IGF1R gene copy number of more than 2, 3, 4, 5, 10, 20, or more) or an increase in IGF1R gene copy number relative to a control (such as a non-neoplastic sample or a reference value) indicates a good prognosis, such as an increase in the likelihood of survival, for the subject. In contrast, no substantial change or a decrease in IGF1R gene copy number (such as an IGF1R gene copy number of about 2 or less) or no substantial change or a decrease in IGF1R gene copy number relative to a control (such as a non-neoplastic sample or a reference value) indicates a poor prognosis, such as a decrease in the likelihood of survival, for the subject.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

We claim:

1. A method for producing nucleic acid tags, comprising:
selecting a prospect nucleic acid sequence from a first genomic sequence, the first genomic sequence corresponding to genomic DNA for a divergent organism;
separating the prospect nucleic acid sequence into a plurality of segment sequences;
comparing the plurality of segment sequences to a second genomic sequence, the second genomic sequence corresponding to genomic DNA for an organism of interest;
selecting a plurality of segment sequences not homologous to any region of the second genomic sequence from the plurality of segment sequences;
preparing a plurality of test oligonucleotides corresponding to the plurality of segment sequences not homologous to any region of the second genomic sequence;
testing hybridization of the plurality of test oligonucleotides against the genomic DNA for the organism of interest;
selecting a plurality of tag sequences identified in the hybridization testing as from the genomic DNA for the organism of interest; and
preparing the nucleic acid tags using one or more of the plurality of nucleic acid tag sequences identified in the hybridization testing as uniquely distinct from the genomic DNA for the organism of interest.

2. The method of claim 1, wherein the plurality of segment sequences are about 20-500 nucleotides (nt).

3. The method of claim 1, wherein the plurality of segment sequences overlap by at least about 10 nt.

4. The method of claim 1, wherein comparing the plurality of segment sequences to the second genomic sequence and/or selecting the plurality of segment sequences not homologous to any region of the second genome from the plurality of segment sequences is done in silico.

5. The method of claim 1, wherein preparing the nucleic acid tags includes synthesizing the nucleic acid tags using oligonucleotide synthesis.

6. The method of claim 5, wherein synthesizing the nucleic acid tags using oligonucleotide synthesis includes using solid-phase oligonucleotide synthesis.

7. The method of claim 6, wherein preparing the nucleic acid tags includes joining the plurality of nucleic acid tag sequences identified in the hybridization testing as uniquely distinct using a joining method selected from the group consisting of enzymatically joining by using a ligase in a ligation reaction, enzymatically joining by using a recombinase in a recombination reaction, chemically joining by using modified nucleotides, and joining by using an amplification reaction.

8. The method of claim 7, wherein the joining method uses a ligase in a ligation reaction.

9. The method of claim 6, wherein preparing the nucleic acid tags includes introducing the plurality of nucleic acid tag sequences identified in the hybridization testing as uniquely distinct into a vector and replicating.

10. The method of claim 1, wherein separating the prospect nucleic acid sequence into a plurality of segment sequences includes eliminating those segment sequences not having G/C nucleotide content between about 30% and 70%.

11. The method of claim 1, wherein testing hybridization of the plurality of test oligonucleotides to the genomic DNA for the organism of interest includes using an array of the plurality of test oligonucleotides.

12. The method of claim 11, wherein testing hybridization of the plurality of test oligonucleotides to the genomic DNA for the organism of interest includes establishing a mathematical model for hybridization scores of total genomic DNA and blocking DNA and establishing one or more predetermined cutoffs.

13. The method of claim 1, wherein the organism of interest is human.

14. The method of claim 1, wherein the divergent organism is an organism having less than about 95% sequence homology with the organism of interest.

15. The method of claim 1, wherein the divergent organism is selected from the group consisting of Oryza, Arabidopsis, and Drosophila.

16. The method of claim 1, wherein the plurality of nucleic acid tag sequences identified in the hybridization testing as uniquely distinct are at least 50 nucleotides in length.


SEQUENCE LISTING (1 sequence)

SEQ ID NO: 1 - DNA, 970 bp, Homo sapiens
  misc_feature (662)..(730): N is A, C, G, or T (masked repetitive region)
gatccaaccttcatggtataaacagacataggtccccggaaataggatgctactatgtgaaaaataaatgggtaaaccataaaagagtaagcatttaccaaaaaaagactgtgttaaacccaagtaagattattttaaactagaagaaactaagataatgcaaattaacaagcttgcctgtctcactttctccactccacactcagcccaccactaaccagatgaacagagcttgagggcaacattatctcaattacagaagattagaaattacaattatttttgtatatctgacttttagcatgtgtatttgaccctataggaccatcattaaataaatgaatctatactattatatggcattacccatgtaagaggtgaattgtaaacccttgcattctagaggctgtactcatgtgacttttgatttaggatcattctgcaaggttaaaaatatgtttggggtatttctcccaagtggcagttgtagcttcttgggaggagaaatgaacaactccaagatcttctcccaggaccactgatgtagcccatgtattaagtcagcccatctaaagcataacatccaaatttaagacaatccatccagttagttctcttgttgtggtagcactcaacatgtaattttatgtatacaaataatnnnnnnnnnnnnnnnnnnnnnnnnnnnnnnnnnnnnnnnnnnnnnnnnnnnnnnnnnnnnnnnnnnnnnggannnnnnnnnnnnnnnnnnnnnnnnnnnnnnnnnnnnnnnnnnnnnnnnnnnnnnnnnnnnnnnnnnnnnnnnnnnnnnnnnnnnntcagccagaagaacaaaacttaaaaaaaaaaatccatcctggctttcaacttcatgtccccaccatgaccatcatcacaactttcaccttactctttttattccacatatactagccaatttgagtgacttgctccagttaggtggtatcac